(19) United States
(12) Patent Application Publication
(10) Pub. No.: US 2024/0262916 A1
(43) Pub. Date: Aug. 8, 2024

(54) METHODS AND COMPOSITIONS FOR TREATING CARDIOVASCULAR DISEASE

(71) Applicant: METANOIA BIO INC., Beverly Hills, CA (US)

(72) Inventors: Slavica TUDZAROVA-TRAJKOVSKA, Beverly Hills, CA (US); Oppel GREEFF, Beverly Hills, CA (US); Abraham VAN WYK, Beverly Hills, CA (US)

(21) Appl. No.: 18/561,127

(22) PCT Filed: May 16, 2022
(86) PCT No.: PCT/US2022/029395

§ 371 Date: Nov. 15, 2023

Related U.S. Application Data
(60) Provisional application No. 63/189,206, filed on May 16, 2021.

(51) Int. Cl. C07K 16/28; A61K 31/196; A61K 31/198; A61K 31/245; A61K 31/26; A61K 31/352; A61K 31/357; A61K 31/37; A61K 31/381; A61K 31/403; A61K 31/41; A61K 31/416; A61K 31/4196; A61K 31/422; A61K 31/4245; A61K 31/427; A61K 31/4709; A61K 31/496; A61K 31/498; A61K 31/501; A61K 31/519; A61K 31/661; A61K 38/06; A61K 38/08; C12N 15/113

(52) U.S. Cl. CPC C07K 16/2857; A61K 31/196; A61K 31/198; A61K 31/245; A61K 31/26; A61K 31/352; A61K 31/357; A61K 31/37; A61K 31/381; A61K 31/403; A61K 31/41; A61K 31/416; A61K 31/4196; A61K 31/422; A61K 31/4245; A61K 31/427; A61K 31/4709; A61K 31/496; A61K 31/498; A61K 31/501; A61K 31/519; A61K 31/661; A61K 38/06; A61K 38/08; C12N 15/1138; C07K 2317/569; C12N 2310/11

(57) ABSTRACT

The disclosure provides methods and compositions for treating cardiovascular disease with HIF1-a Pathway Inhibitors and PFKFB3 Inhibitors. Exemplary cardiovascular diseases ns treated using the provided methods and compositions include acute coronary syndrome, coronary artery disease, myocardial infarction, coronary heart disease, carditis or cardiomyopathy, an ischemic cardiovascular disease, heart failure, stroke, peripheral vascular disease peripheral arterial disease, and ischemia/reperfusion injury.

### BACKGROUND

The disclosure relates to the field of cardiovascular disease. Specifically, the disclosure relates to methods and compositions for treating cardiovascular disease such as, acute coronary syndrome, coronary artery disease, myocardial infarction, coronary heart disease, carditis or cardiomyopathy, an ischemic cardiovascular disease, heart failure, stroke, peripheral vascular disease peripheral arterial disease, and ischemia/reperfusion injury.

Cardiovascular diseases are one of the leading causes of morbidity and mortality worldwide and are projected to remain the leading cause of global mortality over the next decade and beyond. Cardiovascular diseases affect not only cardiovascular disease patients, but also pose a serious health problem for rising numbers of individuals who suffer from metabolic disorders, such as obesity and/or diabetes, which frequently lead to increased cardiovascular risk.

After heart failure due to an occluded artery or another cause of ischemia, the myocardium becomes akinetic or dyskinetic during ischemia. After reperfusion, the contractile function of the heart gradually recovers over the course of a few days and viable myocardial cells are again observed. Previous studies have shown that this “stunning” of the myocardium also occurs in animal models of myocardial infarction (MI). The mechanism(s) responsible for stunned myocardium are thought to be associated with damage from reactive oxygen species (ROS), which occurs in the first few minutes of reperfusion, and also, an altered calcium flux, which desensitizes the myofilaments and may result in decreased myofilament responsiveness. Additionally, stunning is thought to be linked to sarcoplasmic reticulum dysfunction and the consequent calcium overload, which may lead to an uncoupling of the excitation-contraction response.

Type-2 diabetes (T2D) and obesity are risk factors for incident heart failure while increasing the risk of morbidity and mortality in patients with established disease. Trends in the prevalence of patients that suffer from both T2D, obesity and heart failure have been growing. In addition, conditions leading to insulin resistance prior onset of sincere type-2 diabetes T2D produce hyperamilynemia as a result of increased secretion of amylin or islet amyloid pancreatic polypeptide from pancreatic β-cells into circulation. Amylin can be deposited in various organs including the heart where its deposition leads to activation of aberrant calcium oscillations and HIF1-α-PFKFB3 pathway.

Given the ever-increasing number of individuals afflicted with cardiovascular disease and their impact on a global scale, treatment methods that reduce or alleviate cardiovascular disease and are urgently needed.

### BRIEF SUMMARY

The disclosure provides methods and compositions for treating cardiovascular disease such as, acute coronary syndrome, coronary artery disease, myocardial infarction, coronary heart disease, carditis or cardiomyopathy, an ischemic cardiovascular disease, heart failure, stroke, peripheral vascular disease peripheral arterial disease, and ischemia/reperfusion injury. Morae particularly, the disclosure provides methods of treating cardiovascular disease that comprise administering an effective amount of a HIF1-α Pathway Inhibitor or a HIF1-α Inhibitor and an PFKFB3 inhibitor to the subject having or at risk of having the cardiovascular disease.

Hypoxia-inducible factor (HIF1-α) and its transcriptional target 6-phosphofructo-2-kinase/fructose-2,6-biphosphatase 3 (PFKFB) are activated in the cardiomyocytes in the heart, specifically after ischemia-reperfusion injury. The HIF1-α and PFKFB3 pathway is implicated in the mediation of oxidative stress, calcium uptake by the sarcoplasmatic reticulum, and the dysfunction of the sarcoplasmatic reticulum under excessive stress.

When activated, HIF1-α-PFKFB3 signaling provides entrapment of injured cardiomyocytes rendering them resistant to the tissue quality control that eliminates injured cells. This tissue quality control, known as cell competition, guards myocardium from the accumulation of injured, stressed and/or dysfunctional cardiomyocytes.

The accumulation of injured cardiomyocytes that survive cell competition by metabolically switching to aerobic glycolysis via the HIF1-α/PFKFB3 pathway contributes to heart failure, particularly under conditions that mitigate cell competition, such as obesity and T2D.

The inventors have surprisingly discovered that the combination of a HIF1-α inhibitor and a PFKFB3 inhibitor is able to mitigate and possibly even reverse the damage caused by cardiovascular disease and heart failure. Without being limited by theory, it is believed that in the context of heart failure, the disclosed methods induce the regeneration of functional cardiomyocytes after purging of injured cardiomyocytes and reestablishing homeostatic tissue quality control by cell competition.

In some embodiments, the provided methods and compositions treat a chronic cardiovascular disease. In some embodiments, the provided methods and compositions treat an acute cardiovascular disease.

In some embodiments, the disclosure provides:
[1] a method of treating a cardiovascular disease in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and wherein the PFKFB3 inhibitor does not inhibit PI3K/AKT/mTOR pathway or HIF1-α.
[2] the method of [1], wherein the subject is administered an effective amount of the HIF1-α Pathway Inhibitor and the PFKFB3 inhibitor;
[3] the method of [1], wherein the subject is administered an effective amount of the HIF1-α Pathway Inhibitor and wherein the subject has previously been administered the PFKFB3 Inhibitor;
[4] the method of [1], wherein the subject is administered an effective amount of the PFKFB3 Inhibitor and wherein the subject has previously been administered the HIF1-α Pathway Inhibitor;
[5] the method of any one of [1]-[4], wherein the method of any one of 1(a)-1(c) is administered as a prophylactic treatment for the cardiovascular disease;
[6] the method of any one of [1]-[4], wherein the subject has or is at risk of having the cardiovascular disease;
[7] the method of any one of [1]-[4], wherein the subject has or has been diagnosed as having the cardiovascular disease;
[8] the method of any one of [1]-[7], wherein the cardiovascular disease is acute coronary syndrome, coronary artery disease, myocardial infarction, coronary heart disease, carditis or cardiomyopathy, an ischemic cardiovascular disease, heart failure, stroke, peripheral vascular disease peripheral arterial disease, and ischemia/reperfusion injury;
[9] the method of any one of [1]-[8], wherein the cardiovascular disease is ischemic cardiovascular disease such as myocardial ischemia;
[10] the method of any one of [1]-[8], wherein the cardiovascular disease is a peripheral vascular disease or peripheral arterial disease;
[11] the method of any one of [1]-[8], wherein the cardiovascular disease is carditis or cardiomyopathy;
[12] the method of any one of [1]-[8], wherein the cardiovascular disease is ischemia or ischemia/reperfusion injury;
[13] the method of any one of [1]-[12], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, MiRNA, dsRNA, ssRNA, and shRNA), a peptibody, a nanobody, a HIF1-α Pathway binding polypeptide, or a small molecule HIF1-α Pathway Inhibitor;
[14] the method of any one of [1]-[13], wherein the administered HIF1-α Pathway Inhibitor is silibinin, PX-478 or YC-1, or a salt thereof;
[15] the method of any one of [1]-[14], wherein the administered HIF1-α Pathway Inhibitor is ganetespib (ST-9090), phenethyl isothiocyanate, or BAY-87-2243, or a salt thereof;
[16] the method of any one of [1]-[15], wherein the administered HIF1-α Pathway Inhibitor is a HIF1-α Inhibitor;
[17] the method of [16], wherein the HIF1-α Inhibitor is a antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor;
[18] the method of or [17], wherein the administered HIF1-α Inhibitor is antisense oligonucleotide EZN-2968 or nanobody AG-1, AG-2, AG-3, AG-4, AG-5, VHH212, or AHPC;
[19] the method of any one of [1]-[18], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[20] the method of any one of [1]-[19], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[21] the method of any one of [1]-[20], wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[22] the method of any one of [1]-[21], wherein the HIF1-α Pathway Inhibitor and the PFKFB3 inhibitor are co-administered to the subject;
[23] the method of any one of [1]-[22], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous;
[24] the method of any one of [1]-[23], wherein the HIF1-α Pathway Inhibitor and the PFKFB3 inhibitor are administered before the onset of one or more symptoms of the cardiovascular disease;
[25] the method of any one of [1]-[24], wherein treating the cardiovascular disease comprises delaying the onset of the cardiovascular disease;
[26] the method of any one of [1]-[23], wherein the HIF1-α Pathway Inhibitor and the PFKFB3 inhibitor are administered after the onset of one or more symptoms of the cardiovascular disease;
[27] the method of [26], wherein the method results in one or more symptoms of the cardiovascular disease are reduced in the subject administered the HIF1-α Pathway Inhibitor and the PFKFB3 inhibitor compared to in the subject prior to treatment;
[28] the method of [27], wherein the one or more reduced symptoms of the cardiovascular disease is indicated by: reduction in apoptosis/destruction (i.e., loss of) of cardiovascular cells and/or tissue (e.g., endothelial cells, cardiomyocytes, and heart); increase in survival and/or function of cardiovascular cells and/or tissue (e.g., endothelial cells, cardiomyocytes, and heart); reduction in long-term damage to cardiovascular cells/tissue and/or to surrounding cells/tissue; decrease of the inflammation in cardiovascular cells/tissues; reduction in the oxidative stress in cardiovascular cells/tissues; or the reduction of levels of serum biomarkers in the subject, such as, TNFα, IL-1β, IL6, MCP1, IL8, PAF, caspase-3, MMP-2, MMP-9, endothelin-1, leukotrienes B4 and C4; ICAM-1, VCAM-1 PECAM-1, creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, transferrin and/or hyperintense acute reperfusion injury marker (HARM);
[29] the method of or [28], wherein the one or more of the reduction in apoptosis/destruction (i.e., loss of) of cardiovascular cells and/or tissue (e.g., endothelial cells, cardiomyocytes, and heart); increase in survival and/or function of cardiovascular cells and/or tissue (e.g., endothelial cells, cardiomyocytes, and heart); reduction in long-term damage to cardiovascular cells/tissue and/or to surrounding cells/tissue; decrease of the inflammation in cardiovascular cells/tissues; reduction in the oxidative stress in cardiovascular cells/tissues are reduced by at least 10%, at least 20%, at least 30%, at least 40%, or at least 50% compared to in the subject prior to treatment with the HIF1-α Pathway Inhibitor and the PFKFB3 inhibitor;
[30] the method according to any one of [27]-[29], wherein the serum levels of at least 1, 2, 3, 4, or 5 of: TNFα, IL-1β, IL6, MCP1, IL8, PAF, caspase-3, MMP-2, MMP-9, endothelin-1, leukotrienes B4 and C4; ICAM-1, VCAM-1 PECAM-1, creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin and/or HARM are reduced by at least least 20%, compared to in the subject prior to treatment with the HIF1-α Pathway Inhibitor and the PFKFB3 inhibitor;
[31] the method according to any one of [27]-[30], wherein the serum levels of at least 1, 2, 3, 4, or 5 of: TNFα, IL-1β, IL6, MCP1, IL8, PAF, caspase-3, MMP-2, MMP-9, endothelin-1, leukotrienes B4 and C4; ICAM-1, VCAM-1 PECAM-1, and/or HARM are reduced by at least 30%, compared to in the subject prior to treatment with the HIF1-α Pathway Inhibitor and the PFKFB3 inhibitor;
[32] the method of any one of [1]-[31], which further comprises administering an additional therapeutic agent to the subject;
[33] a method of treating a Acute Coronary Syndrome in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and
   wherein the PFKFB3 inhibitor does not inhibit PI3K/AKT/mTOR pathway or HIF1-α;
[34] the method of [33], wherein the subject is administered an effective amount of the HIF1-α Pathway Inhibitor and the PFKFB3 inhibitor;
[35] the method of [33], wherein the subject is administered an effective amount of the HIF1-α Pathway Inhibitor and wherein the subject has previously been administered the PFKFB3 Inhibitor;
[36] the method of [33], wherein the subject is administered an effective amount of the PFKFB3 Inhibitor and wherein the subject has previously been administered the HIF1-α Pathway Inhibitor;
[37] the method of any one of [33]-[36], wherein the method of any one of 1(a)-1(c) is administered as a prophylactic treatment for the Acute Coronary Syndrome;
[38] the method of any one of [33]-[36], wherein the subject has or is at risk of having the Acute Coronary Syndrome;
[39] the method of any one of [33]-[36], wherein the subject has or has been diagnosed as having the Acute Coronary Syndrome;
[40] the method of any one of [33]-[39], wherein the Acute Coronary Syndrome is selected from: unstable angina or myocardial infarction (MI) (e.g., Non-ST-segment elevation MI (NSTEMI) and ST-segment elevation MI (STEMI);
[41] the method of [40], wherein the Acute Coronary Syndrome is unstable angina;
[42] the method of [40], wherein the Acute Coronary Syndrome is myocardial infarction;
[43] the method of [40], wherein the Acute Coronary Syndrome is Non-ST-segment elevation MI;
[44] the method of [40], wherein the Acute Coronary Syndrome is ST-segment elevation MI (STEMI);
[45] the method of any one of [33]-[44], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor;
[46] the method of any one of [33]-[45], wherein the administered HIF1-α Pathway Inhibitor is silibinin, PX-478 or YC-1, or a salt thereof;
[47] the method of any one of [33]-[45], wherein the administered HIF1-α Pathway Inhibitor is ganetespib (ST-9090), phenethyl isothiocyanate, or BAY-87-2243, or a salt thereof;
[48] the method of any one of [33]-[47], wherein the administered HIF1-α Pathway Inhibitor is a HIF1-α Inhibitor;
[49] the method of [48], wherein the HIF1-α Inhibitor is an antibody or antigen-binding antibody fragment (e.g., a single chain antibody, a single-domain antibody (e.g., a fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor;
[50] the method of [48 or 49], wherein the administered HIF1-α Inhibitor is Antisense oligonucleotide EZN-2968 or nanobody AG-1, AG-2, AG-3, AG-4, AG-5, VHH212, or AHPC;
[51] the method of any one of [33]-[50], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[52] the method of any one of [33]-[51], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[53] the method of any one of [33]-[51], wherein the administered PFKFB3 Inhibitor; (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[54] the method of any one of [33]-[53], wherein the HIF1-α Pathway Inhibitor and the PFKFB3 inhibitor are co-administered to the subject;
[55] the method of any one of [33]-[54], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.
[56] the method of any one of [33]-[55], wherein the HIF1-α Pathway Inhibitor and the PFKFB3 inhibitor are administered before the onset of one or more symptoms of the Acute Coronary Syndrome.
[57] the method of any one of [33]-[56], wherein treating the Acute Coronary Syndrome comprises delaying the onset of the Acute Coronary Syndrome.
[58] the method of any one of [33]-[57], wherein the HIF1-α Pathway Inhibitor and the PFKFB3 inhibitor are administered after the onset of one or more symptoms of the Acute Coronary Syndrome.
[59] the method of any one of [33]-[58], wherein the method results in reduction in one or more symptoms of the Acute Coronary Syndrome in the subject administered the HIF1-α Pathway Inhibitor and the PFKFB3 inhibitor compared to in the subject prior to treatment.
[60] the method of [59], wherein the one or more reduced symptoms of the Acute Coronary Syndrome is indicated by:
   (a) reduction in angina, chest pain nausea or vomiting, indigestion, dyspnea; sudden, diaphoresis; lightheadedness, dizziness or fainting; unusual fatigue; restlessness or apprehension;
   (b) a normalized ECG (e.g., reversion of changes in the Q wave and ST segment to normal) or
   (c) reduced levels of plasma concentrations of cardiac enzymes or other biomarkers (e.g., creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin;
[61] the method of or [60], wherein the one or more symptoms of the Acute Coronary Syndrome are reduced by at least 10%, at least 20%, at least 30%, at least 40%, or at least 50% compared to in the subject prior to treatment with the HIF1-α Pathway Inhibitor and the PFKFB3 inhibitor;
[62] the method according to any one of [59]-[61], wherein at least one of the biomarkers creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin is reduced compared to in the subject prior to treatment with the HIF1-α Pathway Inhibitor and the PFKFB3 inhibitor;
[63] the method according to any one of [59]-[62], wherein at least one of the behavioral reflexes; cognitive skills, balance, coordination, and cognitive skills of the subject are improved by at least 10%, at least 20%, at least 30%, at least 40%, or at least 50% compared to in the subject prior to treatment with the HIF1-α Pathway Inhibitor and the PFKFB3 inhibitor;
[64] the method of any one of [33]-[63], which further comprises administering an additional therapeutic agent to the subject.
[65] a method of treating myocardial infarction in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and
   wherein the PFKFB3 inhibitor does not inhibit PI3K/AKT/mTOR pathway or HIF1-α.
[66] the method of [65], wherein the subject is administered an effective amount of the HIF1-α Pathway Inhibitor and the PFKFB3 inhibitor;
[67] the method of [65], wherein the subject is administered an effective amount of the HIF1-α Pathway Inhibitor and wherein the subject has previously been administered the PFKFB3 Inhibitor;
[68] the method of [65], wherein the subject is administered an effective amount of the PFKFB3 Inhibitor and wherein the subject has previously been administered the HIF1-α Pathway Inhibitor;
[69] the method of any one of [65]-[68], wherein the method of any one of 1(a)-1(c) is administered as a prophylactic treatment for myocardial infarction;
[70] the method of any one of [65]-[68], wherein the subject has or is at risk of having myocardial infarction;
[71] the method of any one of [65]-[68], wherein the subject has or has been diagnosed as having myocardial infarction (e.g., Non-ST-segment elevation MI (NSTEMI) or ST-segment elevation MI (STEMI));
[72] the method of any one of [65]-[71], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor;
[73] the method of any one of [65]-[72], wherein the administered HIF1-α Pathway Inhibitor is silibinin, PX-478 or YC-1, or a salt thereof;
[74] the method of any one of [65]-[72], wherein the administered HIF1-α Pathway Inhibitor is ganetespib (ST-9090), phenethyl isothiocyanate, or BAY-87-2243, or a salt thereof;
[75] the method of any one of [65]-[72], wherein the administered HIF1-α Pathway Inhibitor is a HIF1-α Inhibitor.
[76] the method of [75], wherein the HIF1-α Inhibitor is an antibody or antigen-binding antibody fragment (e.g., a single chain antibody, a single-domain antibody (e.g., a fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor;
[77] the method of or [76], wherein the administered HIF1-α Inhibitor is Antisense oligonucleotide EZN-2968 or nanobody AG-1, AG-2, AG-3, AG-4, AG-5, VHH212, or AHPC;
[78] the method of any one of [65]-[77], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[79] the method of any one of [65]-[78], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[80] the method of any one of [65]-[78], wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[81] the method of any one of [65]-[80], wherein the HIF1-α Pathway Inhibitor and the PFKFB3 inhibitor are co-administered to the subject;
[82] the method of any one of [65]-[81], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.
[83] the method of any one of [65]-[82], wherein the HIF1-α Pathway Inhibitor and the PFKFB3 inhibitor are administered before the onset of one or more symptoms of myocardial infarction.
[84] the method of any one of [65]-[83], wherein treating myocardial infarction comprises delaying the onset of myocardial infarction.
[85] the method of any one of [65]-[84], wherein the HIF1-α Pathway Inhibitor and the PFKFB3 inhibitor are administered after the onset of one or more symptoms of myocardial infarction.
[86] the method of any one of [65]-[85], wherein the method results in reduction in one or more symptoms of myocardial infarction the subject administered the HIF1-α Pathway Inhibitor and the PFKFB3 inhibitor compared to in the subject prior to treatment.
[87] the method of [86], wherein the one or more reduced symptoms of myocardial infarction is indicated by:
   (a) reduced angina, chest pain; nausea or vomiting, indigestion, dyspnea; diaphoresis; lightheadedness, dizziness or fainting; unusual fatigue; and restlessness;
   (b) normalized ECG (e.g., reversion of changes in the Q wave and ST segment to normal);
   (c) reduced apoptosis/destruction (i.e., loss of) of or injury to cardiomyocyte cells and/or tissue (e.g., heart); increased survival and/or function of cardiomyocytes and the heart; reduced long-term damage to cardiomyocytes and surrounding cells/tissue; decrease of the inflammation in cardiovascular cells/tissues; reduction in the oxidative stress in cardiovascular cells/tissues; and increased survival/survival time; and/or
   (d) reduced levels reduced levels of plasma MI biomarkers (e.g., creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin;
[88] the method of or [87], wherein the one or more symptoms of myocardial infarction are reduced by at least 10%, at least 20%, at least 30%, at least 40%, or at least 50% compared to in the subject prior to treatment with the HIF1-α Pathway Inhibitor and the PFKFB3 inhibitor;
[89] the method according to any one of [86]-[88], wherein at least one of the plasma MI biomarkers creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin is improved compared to in the subject prior to treatment with the HIF1-α Pathway Inhibitor and the PFKFB3 inhibitor;
[90] the method according to any one of [86]-[89], wherein at least 1, 2, 3, 4, or 5 plasma MI biomarkers creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin are reduced by at least 20%, at least 30%, at least 40%, or at least 50% compared to in the subject prior to treatment with the HIF1-α Pathway Inhibitor and the PFKFB3 inhibitor;
[91] the method of any one of [65]-[90], which further comprises administering an additional therapeutic agent to the subject;
[92] a method of treating heart failure in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and
   wherein the PFKFB3 inhibitor does not inhibit PI3K/AKT/mTOR pathway or HIF1-α;
[93] the method of [92], wherein the subject is administered an effective amount of the HIF1-α Pathway Inhibitor and the PFKFB3 inhibitor;
[94] the method of [92], wherein the subject is administered an effective amount of the HIF1-α Pathway Inhibitor and wherein the subject has previously been administered the PFKFB3 Inhibitor;
[95] the method of [92], wherein the subject is administered an effective amount of the PFKFB3 Inhibitor and wherein the subject has previously been administered the HIF1-α Pathway Inhibitor;
[96] the method of any one of [92]-[95], wherein the method of any one of [92(a)-(c)] is administered as a prophylactic treatment for heart failure;
[97] the method of any one of [92]-[95], wherein the subject has or is at risk of having heart failure;
[98] the method of any one of [92]-[95], wherein the subject has or has been diagnosed as having heart failure;
[99] the method of any one of [92]-[98], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor;
[100] the method of any one of [92]-[99], wherein the administered HIF1-α Pathway Inhibitor is silibinin, PX-478 or YC-1, or a salt thereof;
[101] the method of any one of [92]-[99], wherein the administered HIF1-α Pathway Inhibitor is ganetespib (ST-9090), phenethyl isothiocyanate, or BAY-87-2243, or a salt thereof;
[102] the method of any one of [92]-[99], wherein the administered HIF1-α Pathway Inhibitor is a HIF1-α Inhibitor;
[103] the method of [102], wherein the HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor;
[104] the method of or [103], wherein the administered HIF1-α Inhibitor is Antisense oligonucleotide EZN-2968 or nanobody AG-1, AG-2, AG-3, AG-4, AG-5, VHH212, or AHPC;
[105] the method of any one of [92]-[104], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[106] the method of any one of [92]-[105], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[107] the method of any one of [92]-[105], wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[108] the method of any one of [92]-[107], wherein the HIF1-α Pathway Inhibitor and the PFKFB3 inhibitor are co-administered to the subject;
[109] the method of any one of [92]-[108], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous;
[110] the method of any one of [92]-[109], wherein the HIF1-α Pathway Inhibitor and the PFKFB3 inhibitor are administered before the onset of one or more symptoms of heart failure;
[111] the method of any one of [92]-[110], wherein treating heart failure comprises delaying the onset of heart failure;
[112] the method of any one of [92]-[111], wherein the HIF1-α Pathway Inhibitor and the PFKFB3 inhibitor are administered after the onset of one or more symptoms of heart failure;
[113] the method of any one of [92]-[112], wherein the method results in reduction in one or more symptoms of heart failure the subject administered the HIF1-α Pathway Inhibitor and the PFKFB3 inhibitor compared to in the subject prior to treatment;
[114] the method of [113], wherein the one or more reduced symptoms of heart failure is indicated by: (a) reduction in shortness of breath, fatigue, weakness, leg swelling, exercise intolerance, elevations in heart and respiratory rates, rales (an indication of fluid in the lungs), edema, jugular venous distension, and an enlarged heart; or (b) reduction of at least one serum biomarker for HF (e.g., plasma hsCRP, IL-1beta, and IL-6, and B-type Natriuretic Peptide (BNP));
[115] the method of or [114], wherein at least one of the following symptoms is improved in the subject compared to prior to treatment with the HIF1-α Pathway Inhibitor and the PFKFB3 inhibitor: shortness of breath, fatigue, weakness, leg swelling, exercise intolerance, elevations in heart and respiratory rates, rales (an indication of fluid in the lungs), edema, jugular venous distension, and an enlarged heart;
[116] the method according to any one of [113]-[115], wherein at least one of the one of the biomarkers hsCRP, IL-1beta, and IL-6, and B-type Natriuretic Peptide (BNP) is reduced at least 10%, at least 20%, at least 30%, at least 40%, or at least 50% compared to in the subject prior to treatment with the HIF1-α Pathway Inhibitor and the PFKFB3 inhibitor;
[116] the method according to any one of [113]-[116], wherein at least 1, 2, or 3 of the biomarkers hsCRP, IL-1beta, and IL-6, and B-type Natriuretic Peptide (BNP) is reduced at least 20%, at least 30%, at least 40%, or at least 50% compared to in the subject prior to treatment with the HIF1-α Pathway Inhibitor and the PFKFB3 inhibitor;
[118] the method of any one of [92]-[117], which further comprises administering an additional therapeutic agent to the subject;
[119] a method of treating stroke in a subject in need thereof comprising:
   (a) administering an effective amount of a HIF1-α Pathway Inhibitor and an PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and
   wherein the PFKFB3 inhibitor does not inhibit PI3K/AKT/mTOR pathway or HIF1-α;
[120] the method of [119], wherein the subject is administered an effective amount of the HIF1-α Pathway Inhibitor and the PFKFB3 inhibitor;
[121] the method of [119], wherein the subject is administered an effective amount of the HIF1-α Pathway Inhibitor and wherein the subject has previously been administered the PFKFB3 Inhibitor;
[122] the method of [119], wherein the subject is administered an effective amount of the PFKFB3 Inhibitor and wherein the subject has previously been administered the HIF1-α Pathway Inhibitor;
[123] the method of any one of [119]-[122], wherein the method of any one of [119(a)-(c)] is administered as a prophylactic treatment for stroke;
[124] the method of any one of [119]-[122], wherein the subject has or is at risk of having stroke;
[125] the method of any one of [119]-[122], wherein the subject has or has been diagnosed as having stroke;
[126] the method of any one of [119]-[125], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor;
[127] the method of any one of [119]-[126], wherein the administered HIF1-α Pathway Inhibitor is silibinin, PX-478 or YC-1, or a salt thereof;
[128] the method of any one of [119]-[126], wherein the administered HIF1-α Pathway Inhibitor is ganetespib (ST-9090), phenethyl isothiocyanate, or BAY-87-2243, or a salt thereof;
[129] the method of any one of [119]-[126], wherein the administered HIF1-α Pathway Inhibitor is a HIF1-α Inhibitor;
[130] the method of [129], wherein the HIF1-α Inhibitor is a antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor;
[131] the method of [129 or 130], wherein the administered HIF1-α Inhibitor is Antisense oligonucleotide EZN-2968 or nanobody AG-1, AG-2, AG-3, AG-4, AG-5, VHH212, or AHPC;
[132] the method of any one of [119]-[131], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[133] the method of any one of [119]-[132], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[134] the method of any one of [119]-[132], wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[135] the method of any one of [119]-[134], wherein the HIF1-α Pathway Inhibitor and the PFKFB3 inhibitor are co-administered to the subject;
[136] the method of any one of [119]-[135], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous;
[137] the method of any one of [119]-[136], wherein the HIF1-α Pathway Inhibitor and the PFKFB3 inhibitor are administered before the onset of one or more symptoms of stroke;
[138] the method of any one of [119]-[137], wherein treating stroke comprises delaying the onset of stroke;
[139] the method of any one of [119]-[138], wherein the HIF1-α Pathway Inhibitor and the PFKFB3 inhibitor are administered after the onset of one or more symptoms of stroke;
[140] the method of any one of [119]-[139], wherein the method results in reduction in one or more symptoms of stroke the subject administered the HIF1-α Pathway Inhibitor and the PFKFB3 inhibitor compared to in the subject prior to treatment;
[141] the method of [140], wherein the one or more reduced symptoms of stroke is indicated by:
   (a) reduced numbness or weakness in the face, arm, or leg, especially on one side of the body; confusion, trouble speaking, or difficulty understanding speech; trouble seeing in one or both eyes; and/or trouble walking, dizziness, loss of balance, or lack of coordination;
   (b) reduced lesion volume, reduced brain inflammatory levels, increased probability of recovery on the mRS score, and/or reduced cytotoxic edema;
   (c) reduced apoptosis/destruction (i.e., loss of) of or injury to endothelial cells, neural cells, and/or tissue (e.g., neural tissue); increased survival and/or function of vascular endothelial cells and/or neural cells; reduced in long-term damage to vascular endothelial cells, neural cells, and surrounding cells/tissue; decrease of the inflammation in vascular endothelial and/or neural cells/tissues; reduction in the oxidative stress in vascular endothelial and/or neural cells; and increased survival/survival time; or
   (d) reduced levels of serum biomarkers for stroke (e.g., E-selectin, ICAM-1, VCAM, and MCP-1) in the subject;
[142] the method of or [141], wherein the one or more symptoms of stroke are reduced by at least 10%, at least 20%, at least 30%, at least 40%, or at least 50% compared to in the subject prior to treatment with the HIF1-α Pathway Inhibitor and the PFKFB3 inhibitor;
[143] the method according to any one of [140]-[142], wherein at least one of the following symptoms are reduced in the subject compared to in the subject prior to treatment with the HIF1-α Pathway Inhibitor and the PFKFB3 inhibitor: numbness or weakness in the face, arm, or leg; confusion, trouble speaking, or difficulty understanding speech; trouble seeing in one or both eyes; and/or trouble walking, dizziness, loss of balance, or lack of coordination;
[144] the method according to any one of [140]-[143], wherein at least one serum biomarker for stroke selected from: E-selectin, ICAM-1, VCAM, and MCP-1 is reduced by at least 20%, at least 30%, at least 40%, or at least 50% compared to in the subject prior to treatment with the HIF1-α Pathway Inhibitor and the PFKFB3 inhibitor;
[145] the method of any one of [119]-[144], which further comprises administering an additional therapeutic agent to the subject;
[146] a method of treating ischemia or ischemia/reperfusion injury in a subject in need thereof comprising:
   (a) administering an effective amount of a HIF1-α Pathway Inhibitor and a PFKFB3 inhibitor to the subject;
   (b) administering an effective amount of a HIF1-α Pathway Inhibitor to the subject, wherein the subject has previously been administered a PFKFB3 Inhibitor; or
   (c) administering an effective amount of a PFKFB3 Inhibitor to the subject, wherein the subject has previously been administered a HIF1-α Pathway Inhibitor; and
   wherein the PFKFB3 inhibitor does not inhibit PI3K/AKT/mTOR pathway or HIF1-α;
[147] the method of [146], wherein the subject is administered an effective amount of the HIF1-α Pathway Inhibitor and the PFKFB3 inhibitor;
[148] the method of [146], wherein the subject is administered an effective amount of the HIF1-α Pathway Inhibitor and wherein the subject has previously been administered the PFKFB3 Inhibitor;
[149] the method of [146], wherein the subject is administered an effective amount of the PFKFB3 Inhibitor and wherein the subject has previously been administered the HIF1-α Pathway Inhibitor;
[150] the method of any one of [146]-[149], wherein the method of any one of [146(a)-(c)] is administered as a prophylactic treatment for ischemia or ischemia/reperfusion injury;
[151] the method of any one of [146]-[149], wherein the subject has or is at risk of having ischemia or ischemia/reperfusion injury;
[152] the method of any one of [146]-[149], wherein the subject has or has been diagnosed as having ischemia or ischemia/reperfusion injury;
[153] the method of any one of [146]-[153], wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor;
[154] the method of any one of [146]-[153], wherein the administered HIF1-α Pathway Inhibitor is silibinin, PX-478 or YC-1, or a salt thereof;
[155] the method of any one of [146]-[153], wherein the administered HIF1-α Pathway Inhibitor is ganetespib (ST-9090), phenethyl isothiocyanate, or BAY-87-2243, or a salt thereof;
[156] the method of any one of [146]-[153], wherein the administered HIF1-α Pathway Inhibitor is a HIF1-α Inhibitor;
[157] the method of [156], wherein the HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor;
[158] the method of [156] or [157], wherein the administered HIF1-α Inhibitor is Antisense oligonucleotide EZN-2968 or nanobody AG-1, AG-2, AG-3, AG-4, AG-5, VHH212, or AHPC;
[159] the method of any one of [146]-[158], wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor;
[160] the method of any one of [146]-[159], wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof;
[161] the method of any one of [146]-[159], wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof;
[162] the method of any one of [146]-[161], wherein the HIF1-α Pathway Inhibitor and the PFKFB3 inhibitor are co-administered to the subject;
[163] the method of any one of [146]-[162], wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous;
[164] the method of any one of [146]-[163], wherein the ischemia or ischemia/reperfusion injury is due to a condition selected from: infarction, atherosclerosis, thrombosis, thromboembolism, lipid-embolism, bleeding, stent, surgery, angioplasty, end of bypass during surgery, organ transplantation, or total ischemia;
[165] the method of any one of [146]-[163], wherein the ischemia or ischemia/reperfusion injury is ischemia/reperfusion injury is selected from: organ dysfunction, infarct, inflammation, oxidative damage, mitochondrial membrane potential damage, apoptosis, reperfusion-related arrhythmia, cardiac stunning, cardiac lipotoxicity, or ischemia-derived scar formation;
[166] the method of any one of [146]-[165], wherein ischemia/reperfusion injury is due to myocardial infarction;
[167] the method of any one of [146]-[165], wherein the HIF1-α Pathway Inhibitor and the PFKFB3 inhibitor are administered:
   (a) during ischemia or prior to reperfusion;
   (b) during reperfusion; or
   (c) after the ischemia and ischemia/reperfusion;
[168] the method of any one of [146]-[167], wherein treating ischemia or ischemia/reperfusion injury comprises delaying the onset of ischemia or ischemia/reperfusion injury;
[169] the method of any one of [146]-[168], wherein the HIF1-α Pathway Inhibitor and the PFKFB3 inhibitor are administered after the onset of one or more symptoms of ischemia or ischemia/reperfusion injury;
[170] the method of any one of [146]-[169], wherein the method results in reduction in one or more symptoms of ischemia or ischemia/reperfusion injury the subject administered the HIF1-α Pathway Inhibitor and the PFKFB3 inhibitor compared to in the subject prior to treatment;
[171] the method of [170], wherein the one or more reduced symptoms of ischemia or ischemia/reperfusion injury is indicated by:
   (a) reduced apoptosis/destruction (i.e., loss of) of or injury to endothelial cells and/or tissue (e.g., neural tissue); increased survival and/or function of endothelial cells; reduced long-term damage to endothelial cells, and surrounding cells/tissue; decrease of the inflammation in endothelial cells/tissues; reduction in the oxidative stress in endothelial; and increased survival/survival time;
   (b) reduced levels of at least one ischemia/reperfusion biomarker (e.g., caspase-3, MMP-2, MMP-9, endothelin-1, leukotrienes B4 and C4; TNFα, IL1, IL6, IL8, PAF, ICAM-1, VCAM-1 PECAM-1, and HARM; or
   (c) reduced extent of no reflow phenomenon in the subject;
[172] the method of or [171], wherein the one or more symptoms of ischemia or ischemia/reperfusion injury are reduced by at least 10%, at least 20%, at least 30%, at least 40%, or at least 50% compared to in the subject prior to treatment with the HIF1-α Pathway Inhibitor and the PFKFB3 inhibitor;
[173] the method according to any one of [170]-[172], wherein the extent of no reflow phenomenon is reduced in the subject;
[174] the method according to any one of [170]-[173], wherein the level of at least 1, 2, 3, 4, or 5, ischemia/reperfusion biomarker selected from caspase-3, MMP-2, MMP-9, endothelin-1, leukotrienes B4 and C4; TNFα, IL1, IL6, IL8, PAF, ICAM-1, VCAM-1 PECAM-1, and HARM is reduced in the subject by at least 20%, at least 30%, at least 40%, or at least 50% compared to in the subject prior to treatment with the HIF1-α Pathway Inhibitor and the PFKFB3 inhibitor; and/or
[175] the method of any one of [146]-[174], which further comprises administering an additional therapeutic agent to the subject.

### BRIEF DESCRIPTION OF THE DRAWINGS/FIGURES

FIGS. 1A-1E, depict exemplary PFKFB3 small molecule inhibitors.

### DETAILED DESCRIPTION

### Definitions

Unless otherwise defined, all technical and scientific terms used herein have the same meaning as commonly understood by one of ordinary skill in the art to which this disclosure pertains. Although methods and materials similar or equivalent to those described herein can be used in the practice or testing of the provided compositions, suitable methods and materials are described below. Each publication, patent application, patent, and other reference mentioned herein is herein incorporated by reference in its entirety. In case of conflict, the present specification, including definitions, will control. In addition, the materials, methods, and examples are illustrative only and are not intended to be limiting.

Other features and advantages of the disclosed methods and compositions will be apparent from the following disclosure, drawings, and claims.

It is understood that wherever embodiments, are described herein with the language “comprising” otherwise analogous embodiments, described in terms of “containing” “consisting of” and/or “consisting essentially of” are also provided. However, when used in the claims as transitional phrases, each should be interpreted separately and in the appropriate legal and factual context (e.g., in claims, the transitional phrase “comprising” is considered more of an open-ended phrase while “consisting of” is more exclusive and “consisting essentially of” achieves a middle ground).

As used herein, the singular form “a”, “an”, and “the”, include plural forms unless it is expressly stated or is unambiguously clear from the context that such is not intended. The singular form “a”, “an”, and “the” also includes the statistical mean composition, characteristics, or size of the particles in a population of particles (e.g., mean polyethylene glycol molecular weight mean liposome diameter, mean liposome zeta potential). The mean particle size and zeta potential of liposomes in a pharmaceutical composition can routinely be measured using methods known in the art, such as dynamic light scattering. The mean amount of a therapeutic agent in a nanoparticle composition may routinely be measured for example, using absorption spectroscopy (e.g., ultraviolet-visible spectroscopy).

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a nanoparticle composition, “about” may mean+/−10% of the recited value. For instance, a nanoparticle composition including a lipid component having about 40% of a given compound may include 30-50% of the compound.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Recitation of ranges of values herein are merely intended to serve as a shorthand method of referring individually to each separate value falling within the range, unless otherwise indicated herein, and each separate value is incorporated into the specification as if it were individually recited herein.

Where embodiments of the disclosure are described in terms of a Markush group or other grouping of alternatives, the disclosed composition or method encompasses not only the entire group listed as a whole, but also each member of the group individually and all possible subgroups of the main group, and also the main group absent one or more of the group members. The disclosed methods and compositions also envisage the explicit exclusion of one or more of any of the group members in the disclosed compositions or methods.

The terms “antibody” and “antigen-binding antibody fragment” and the like, as used herein, include any protein or peptide containing molecule that comprises at least a portion of an immunoglobulin molecule, such as, but not limited to, at least one complementarity determining region (CDR) of a heavy or light chain or an antigen binding portion thereof.

The term “antibody” also includes fragments, specified portions and variants thereof, including antibody mimetics or comprising portions of antibodies that mimic the structure and/or function of an antibody or specified fragment or portion thereof, including single chain antibodies, single binding domain antibodies and antigen binding antibody fragments.

The term “antibody fragment” refers to a portion of an intact antibody, generally the antigen binding or variable region of an intact antibody. Examples of antibody fragments include, but are not limited to Fab, Fab′, F(ab′)2, single chain (scFv) and Fv fragments, diabodies; linear antibodies; single-chain antibody molecules; single Fab arm “one arm” antibodies and multispecific antibodies formed from antibody fragments, among others. Antibody fragments include any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of an antigen or antigen receptor or binding protein, which can be incorporated into an antibody provided herein.

Antibody fragments can be produced by enzymatic cleavage, synthetic or recombinant techniques, as known in the art. Antibodies can also be produced in a variety of truncated forms using antibody genes in which one or more stop codons have been introduced upstream of the natural stop site. For example, a combination gene encoding a F(ab′)2 heavy chain portion can be designed to include DNA sequences encoding the CH1 domain and/or hinge region of the heavy chain. The various portions of antibodies can be joined together chemically by conventional techniques, or can be prepared as a contiguous protein using genetic engineering techniques.

The terms “nucleic acid” and “oligonucleotide” are used interchangeably herein and refer to at least two nucleotides covalently linked together. In some embodiments the HIF1-α pathway inhibitor and/or PFKFB3 inhibitor administered according to the provided methods is a therapeutic nucleic acid. In some embodiments, the administered nucleic acid is an ENMD-1198, an shRNA, a Dicer substrate (e.g., dsRNA), an miRNA, an anti-miRNA, an antisense molecule, a decoy, or an aptamer, or a plasmid capable of expressing a ENMD-1198, an shRNA, a Dicer substrate, an miRNA, an anti-miRNA, an antisense molecule, a decoy, or an aptamer.

The nucleic acids administered according to the provided methods are preferably single-stranded or double-stranded and generally contain phosphodiester bonds, although in some cases, nucleic acid/oligonucleotide analogs are included that have alternate backbones, comprising, example, for phosphoramide, phosphorothioate, phosphorodithioate, O-methylphosphoroamidiate linkages, and peptide nucleic acid backbones and linkages. Other analog nucleic acids/oligonucleotides include those with positive backbones; non-ionic backbones, and non-ribose backbones. Nucleic acids/oligonucleotides containing one or more carbocyclic sugars are also included within the definition of nucleic acids and oligonucleotides. These modifications of the ribose-phosphate backbone may be done for example, to facilitate the addition of additional moieties such as labels, or to increase the stability and half-life of such molecules in physiological environments. Nucleic acid/oligonucleotide backbones of oligonucleotides used according to the provided methods can range from about 5 nucleotides to about 750 nucleotides. Preferred nucleic acid/oligonucleotide backbones range from about 5 nucleotides to about 500 nucleotides, and preferably from about 10 nucleotides to about 100 nucleotides in length.

The oligonucleotides administered according to the provided methods are polymeric structures of nucleoside and/or nucleotide monomers capable of specifically hybridizing to at least a region of a nucleic acid target. As indicated above, the “nucleic acids” and “oligonucleotides” used according to the provided methods include, but are not limited to, compounds comprising naturally occurring bases, sugars and intersugar (backbone) linkages, non-naturally occurring modified monomers, or portions thereof (e.g., oligonucleotide analogs or mimetics) which function similarly to their naturally occurring counterpart, and combinations of these naturally occurring and non-naturally occurring monomers. As used herein, the term “modified” or “modification” includes any substitution and/or any change from a starting or natural oligomeric compound, such as an nucleic acid. Modifications to nucleic acids encompass substitutions or changes to internucleoside linkages, sugar moieties, or base moieties, such as those described herein and those otherwise known in the art.

As used herein, a “small molecule” refers to an organic compound that is either synthesized via conventional organic chemistry methods (e.g., in a laboratory) or found in nature. Typically, a small molecule is characterized in that it contains several carbon-carbon bonds, and has a molecular weight of less than about 1500 grams/mole. In certain embodiments, small molecules are less than about 1000 grams/mole. In certain embodiments, small molecules are less than about 550 grams/mole. In certain embodiments, small molecules are between about 200 and about 550 grams/mole. In certain embodiments, small molecules exclude peptides (e.g., compounds comprising 2 or more amino acids joined by a peptidyl bond). In certain embodiments, small molecules exclude nucleic acids.

The terms “condition” and “disease” are used interchangeably herein and refer to any condition or disorder that damages, interferes with or dysregulates the normal function of a cell, tissue, or organ.

As used herein, “cardiovascular disease” and “CVD” are terms used to refer to a condition affecting the heart, heart valves, and/or vasculature (e.g., arteries and veins) of the body and encompasses diseases and conditions including, but not limited to coronary artery diseases (CAD) (such as angina and myocardial infarction (commonly known as a heart attack), coronary heart disease (e.g., ischemic heart disease), acute coronary syndrome, angina, heart failure, aortic aneurysm, aortic dissection, iliac or femoral aneurysm, pulmonary embolism, primary hypertension, atrial fibrillation, stroke, transient ischemic attack, systolic dysfunction, diastolic dysfunction, carditis (e.g., endocarditis, myocarditis, acute myocarditis, acute pericarditis and complicated pericarditis), atrial tachycardia, ventricular fibrillation, cardiac allograft rejection arteriopathy, vasculitis, thrombosis, atherosclerosis, atherosclerotic plaque, vulnerable plaque, acute coronary syndrome, acute ischemic attack, sudden cardiac death, cerebrovascular disease, peripheral vascular disease, peripheral artery disease (PAD), and cerebrovascular disease, cardiomyopathy, cardiac dysrhythmias, inflammatory heart disease,

In some embodiments, a subject treated according to the provided methods and compositions is identified as having cardiovascular disease by the presence of one or more of: documented coronary artery disease, documented cerebrovascular disease, documented carotid disease, documented peripheral arterial disease, or combinations thereof. In some embodiments, a subject treated according to the provided methods is identified as having cardiovascular disease if the subject is at least 45 years old and: (a) has one or more stenosis of greater than 50% in two major epicardial coronary arteries; (b) has had a documented prior MI; (c) has been hospitalized for high-risk NSTE ACS with objective evidence of ischemia (e.g., ST-segment deviation and/or biomarker positivity); (d) has a documented prior ischemic stroke; (e) has symptomatic artery disease with at least 50% carotid arterial stenosis; (0 has asymptomatic carotid artery disease with at least 70% carotid arterial stenosis per angiography or duplex ultrasound; (g) has an ankle-brachial index (“ABI”) of less than 0.9 with symptoms of intermittent claudication; and/or (h) has a history of aorto-iliac or peripheral arterial intervention (catheter-based or surgical).

A cardiovascular event, as used herein, refers to the manifestation of an adverse condition in a subject brought on by cardiovascular disease, such as sudden cardiac death or acute coronary syndromes including, but not limited to, myocardial infarction, unstable angina, aneurysm, or stroke. The term “cardiovascular event” can be used interchangeably herein with the term cardiovascular complication. While a cardiovascular event can be an acute condition, it can also represent the worsening of a previously detected condition to a point where it represents a significant threat to the health of the subject, such as the enlargement of a previously known aneurysm or the increase of hypertension to life threatening levels.

As used herein, the term “progression of a cardiovascular disease” refers to the gradual worsening of the disease over time, whereby symptoms and cardiovascular chemical deficits become increasingly more debilitating and/or intense.

As used herein, the term “inhibiting progression of a CVD disease” refers to slowing and/or stopping the progression of symptoms of a cardiovascular disease.

As used herein, “delaying development” of a cardiovascular diseases a disease, such as acute coronary syndrome, coronary artery disease, myocardial infarction, coronary heart disease, carditis, heart failure, stroke, peripheral vascular disease and/or reperfusion injury, means to defer, hinder, slow, retard, stabilize, and/or postpone development of one or more symptoms, of the disease, including decreasing the rate at which the patient's disease progresses (e.g., to shift the patient from rapidly progressing disease to a more slowly progressing disease). This delay can be of varying lengths of time, depending on the history of the disorder and/or the medical profile of the individual being treated. As is evident to one skilled in the art, a sufficient or significant delay can, in effect, encompass prevention, in that the individual does not develop detectable disease. A method that “delays” development of disease is a method that reduces the extent of the disease in a given time frame, when compared to not using the method. Such comparisons are typically based on clinical studies, using a statistically significant number of subjects, although this knowledge can be based upon anecdotal evidence. “Delaying development” can mean that the extent and/or undesirable clinical manifestations are lessened and/or time course of the progression is slowed or lengthened, as compared to not administering the agent. Thus the term also includes, but is not limited to, alleviation of symptoms, diminishment of extent of disease, stabilized (i.e., not worsening) state of disease, delay or slowing of disease progression, and remission (whether partial or total) whether detectable or undetectable.

As used herein an “effective amount” refers to a dosage of an agent sufficient to provide a medically desirable prophylactic and/or therapeutic effect on a cardiovascular disease (e.g., a CVD). The effective amount will vary with the desired outcome, the particular CVD being treated (or prevented), the age and physical condition of the subject being treated, the severity of the condition, the duration of the treatment, the nature of the concurrent or combination therapy (if any), the specific route of administration and like factors within the knowledge and expertise of the health practitioner. An “effective amount” can be determined empirically and in a routine manner, in relation to the stated purpose. A prophylactic and/or therapeutic effect includes, but is not limited to, reduction in apoptosis/destruction (i.e., loss of) of cardiovascular cells and/or tissue; increase in survival and/or function of cardiovascular cells and/or tissue; reduction in long-term damage to cardiovascular cells/tissue and/or to surrounding cells/tissue; decrease of the inflammation in cardiovascular cells/tissues; reduction in the oxidative stress in cardiovascular cells/tissues; and increased survival/survival time.

The terms “subject”, “patient,” “individual,” and “animal” are used interchangeably and refer to mammals such as human patients and non-human primates, as well as experimental animals such as rabbits, rats, and mice, and other laboratory animals. Animals include all vertebrates, e.g., mammals and non-mammals, such as chickens, amphibians, and reptiles. “Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and other members of the class Mammalia known in the art. In a particular embodiment, the patient is a human.

Terms such as “treating.” or “treatment,” “to treat,” or “therapy,” refer to both (a) therapeutic measures that cure, slow down, attenuate, lessen symptoms of, and/or halt progression of a pathologic condition and (b) prophylactic or preventative measures that prevent and/or slow the development of a targeted condition and or its related symptoms

Thus, subjects in need of treatment include those already with the cardiovascular disease; those at risk of having the cardiovascular disease; and those in whom the cardiovascular disease is to be prevented. Subjects can routinely be identified as “having or at risk of having” a cardiovascular disease or another condition referred to herein using medical and diagnostic techniques known in the art. In certain embodiments, a subject is successfully “treated” according to the provided methods if the subject shows, e.g., total, partial, or transient amelioration or elimination of at least one symptom associated with the condition such as the patient showing or feeling a reduction in any one of the symptoms of angina pectoris, fatigue, weakness, breathlessness, leg swelling, rales, heart or respiratory rates, edema or jugular venous distension. The patient may also show greater exercise tolerance, have a smaller heart with improved ventricular and cardiac function, and in general, require fewer hospital visits related to the heart condition. The improvement in cardiovascular function may be adequate to meet the metabolic needs of the patient and the patient may not exhibit symptoms under mild exertion or at rest. Many of these signs and symptoms are readily observable by eye and/or measurable by routine procedures familiar to a physician. Indicators of improved cardiovascular function include increased blood flow and/or contractile function in the treated tissues. As described below, blood flow in a patient can be measured by thallium imaging (as described by Braunwald in Heart Disease, 4th ed., pp. 276-311 (Saunders, Philadelphia, 1992)) or by echocardiography (described in Examples 1 and 5 and in Sahn, D J., et al., Circulation. 58:1072-1083, 1978). Blood flow before and after angiogenic gene transfer can be compared using these methods. Improved heart function is associated with decreased signs and symptoms, as noted above. In addition to echocardiography, one can measure ejection fraction (LV) by nuclear (non-invasive) techniques as is known in the art. Blood flow and contractile function can likewise be measured in peripheral tissues treated according to the present invention.

In other embodiments, the terms “treating,” or “treatment,” “to treat,” or “therapy,” refer to the inhibition of the progression of a cardiovascular disease, either physically by, e.g., stabilization of a discernible symptom, physiologically by, e.g., stabilization of a physical parameter, or both. In other embodiments, the terms “treating,” or “treatment,” “to treat,” or “therapy,” refer to the reduction or alleviation of symptoms, the reduction of inflammation, the inhibition of cell death, and/or the restoration of cell function. Treatment can be with the HIF1-α Pathway Inhibitor and PFKFB3 inhibitor compositions disclosed herein, or in further combination with one or more additional therapeutic agent.

The term “pharmaceutically acceptable carrier” refers to an ingredient in a pharmaceutical formulation, other than an active ingredient, which is nontoxic to a subject. A pharmaceutically acceptable carrier includes, but is not limited to, a buffer, carrier, excipient, stabilizer, diluent, or preservative. Pharmaceutically acceptable carriers can include for example, one or more compatible solid or liquid filler, diluents or encapsulating substances which are suitable for administration to a human or other subject.

“Therapeutic agent(s)” used according to the disclosed methods and compositions can additionally include any agent directed to treat a condition in a subject.

### PFKFB3 Inhibitors

PFKFB3 (6-phosphofructo-2-kinase-fructose-2,6-bisphosphatase 3) is a bifunctional protein that is involved in both the synthesis and degradation of fructose-2,6-bisphosphate, a regulatory molecule that controls glycolysis in eukaryotes and is required for cell cycle progression and the prevention of apoptosis.

In some embodiments, the disclosure provides a method of treating a cardiovascular disease in a subject in need thereof that comprises:
(a) administering an effective amount of a HIF1-α Pathway Inhibitor or a HIF1-α Inhibitor and an PFKFB3 inhibitor to the subject;
(b) administering an effective amount of a HIF1-α Pathway Inhibitor or a HIF1-α Inhibitor to the subject, wherein the subject has previously been administered a PFKFB3 Inhibitor; or
(c) administering an effective amount of a PFKFB3 Inhibitor to the subject wherein the subject has previously been administered a HIF1-α Pathway Inhibitor or a HIF1-α Inhibitor to the subject;
   wherein the PFKFB3 Inhibitor does not inhibit the PI3K/AKT/mTOR pathway or HIF1-α.

The PFKFB3 Inhibitors that can be used according to the provided methods are not particularly limited. In some embodiments, the administered PFKFB3 Inhibitor is an antibody or a PFKFB3-binding antibody (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, miRNA, dsRNA, ssRNA, and shRNA), a peptibody, a nanobody, a PFKFB3 inhibitory binding polypeptide, or a small molecule PFKFB3 Inhibitor.

In some embodiments, the PFKFB3 inhibitor administered according to the provide methods has an IC50 for a PFKFB3 activity/function of 100 μM or lower concentration for a PFKFB3 activity. In some embodiments, the PFKFB3 inhibitor has an IC50 of at least or at most or about 200, 100, 80, 50, 40, 20, 10, 5, or 1 μM, or at least or at most or about 100, 10, or 1 nM, or lower (or any range or value derivable therefrom). In some embodiments, the PFKFB3 inhibitor inhibits the expression of PFKFB3. Assays for determining the ability of a compound to inhibit PFKFB3 activity are known in the art. In some embodiments, the inhibition of PFKFB3 activity or expression is a decrease as compared with a control level or sample. In some embodiments, a functional assay such as an MTT assay, cell proliferation assay, BRDU or Ki67 immunofluorescence assay, apoptosis assay, or glycolysis assay is used to assay for the ability of a composition to inhibit PFKFB3 activity.

In some embodiments, the PFKFB3 Inhibitor administered according to the provided methods is an antibody or a PFKFB3-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), In particular embodiments, the administered PFKFB3 Inhibitor is a nanobody (e.g., a VHH).

In some embodiments, the HIF1-A Inhibitor administered according to the provided methods is a therapeutic nucleic acid. In some embodiments the therapeutic nucleic acid is an aptamer, antisense molecule, ribozyme, a Dicer substrate, miRNA, dsRNA, ssRNA, and shRNA). In particular embodiments, the HIF1-α Inhibitor administered according to the provided methods is an siRNA or an antisense oligonucleotide. In one embodiment, the administered PFKFB3 Inhibitor is EZN-4178.

Representative examples of human PFKFB3 coding sequences are provided in GenBank accession numbers NM_004566.3. NM_001145443.2. NP_001138915.1. NM_001282630.2. NM_001314063.1, NM_001323016.1, NM_001323017.1, and NM_001363545.2. The sequences associated with the each of these Genbank accession numbers is hereby incorporated by reference herein in its entirety for all purposes. Therapeutic nucleic acids that inhibit PFKFB3 activity can routinely be designed and prepared based on each of the above human PFKFB3 transcript sequences using methods known in the art.

The administration of PFKFB3 inhibitory nucleic acids or any ways of inhibiting gene expression of PFKFB3 known in the art are contemplated in certain embodiments of the provided methods. Examples of inhibitory nucleic acid include but are not limited to, antisense nucleic acids such as: small interfering RNA (SiRNA), short hairpin RNA (shRNA), double-stranded RNA, and any other antisense oligonucleotide. Also included are ribozymes or nucleic acids encoding any of the inhibitors described herein. An inhibitory nucleic acid may inhibit the transcription of PFKFB3 or prevent the translation of a PFKFB3 gene transcript in a cell. In some embodiments, the PFKFB3 inhibitory nucleic acid adminstered according to the provided methods is from 16 to 1000 nucleotides in length. In certain embodiments the administered PFKFB3 inhibitory nucleic acid is from 18 to 100 nucleotides long. In certain embodiments the administered PFKFB3 inhibitory nucleic acid at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 nucleotides or any range derivable therefrom.

In some embodiments, the PFKFB3 inhibitory nucleic acid administered according to the provided methods is capable of decreasing the expression of PFKFB3 by at least 10%, 20%, 30%, or 40%, more particularly by at least 50%, 60%, or 70%, and most particularly by at least 75%, 80%, 90%, 95% or more or any range or value in between the foregoing.

In some embodiments, the PFKFB3 inhibitory nucleic acid administered according to the provided methods is between 17 to 25 nucleotides in length and comprises a 5′ to 3′ sequence that is at least 90% complementary to the 5′ to 3′ sequence of a mature PFKFB3 mRNA (e.g., a sequence as disclosed in any one or more of GenBank accession nos. NM_004566.3, NM_001145443.2, NM_001282630.2, NM_001314063.1, NM_001323016.1, NM_001323017.1, and NM_001363545.2). In some embodiments, the administered PFKFB3 inhibitory nucleic acid is 17, 18, 19, 20, 21, 22, 23, 24, or 25 nucleotides in length, or any range derivable therein. In some embodiments, the administered PFKFB3 inhibitory nucleic acid has a sequence (from 5′ to 3′) that is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the corresponding 5′ to 3′ sequence of a mature PFKFB3 mRNA (e.g., a sequence as disclosed in any one or more of GenBank accession nos. NM_004566.3, NM_001145443.2, NM_001282630.2, NM_001314063.1, NM_001323016.1, NM_001323017.1, and NM_001363545.2). One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the PFKFB3 inhibitory nucleic acid administered according to the provided methods is a miRNA. In further embodiments, the administered miRNA is a member selected from: hsa-mir-26b-5p (MIRT028775), hsa-mir-330-3p (MIRT043840), hsa-mir-6779-5p (MIRT454747), hsa-mir-6780a-5p (MIRT454748), hsa-mir-3689c (MIRT454749), hsa-mir-3689b-3p (MIRT454750), hsa-mir-3689a-3p (MIRT454751), hsa-mir-30b-3p (MIRT454752), hsa-mir-1273h-5p (MIRT454753), hsa-mir-6778-5p (MIRT454754), hsa-mir-1233-5p (MIRT454755), hsa-mir-6799-5p (MIRT454756), hsa-mir-7106-5p (MIRT454757), hsa-mir-6775-3p (MIRT454758), hsa-mir-1291 (MIRT454759), hsa-mir-765 (MIRT454760), hsa-mir-423-5p (MIRT454761), hsa-mir-3184-5p (MIRT454762), hsa-mir-6856-5p (MIRT454763), hsa-mir-6758-5p (MIRT454764), hsa-mir-3185 (MIRT527973), hsa-mir-6892-3p (MIRT527974), hsa-mir-6840-5p (MIRT527975), and hsa-mir-6865-3p (MIRT527976).

In some embodiments, the PFKFB3 inhibitor administered according to the provide methods is a small molecule. The administered small molecule PFKFB3 inhibitors may be any small molecules that is determined to inhibit PFKFB3 function or activity. Such small molecules may be determined based on functional assays in vitro or in vivo.

In some embodiments, the PFKFB3 inhibitor small molecules administered according to the provide methods is a small molecule PFKFB3 inhibitory molecules disclosed in U.S. publication nos. 20130059879, 20120177749, 20100267815, 20100267815, and 20090074884, the disclosure of each of which is herein incorporated by reference in its entirety.

In some embodiments, the PFKFB3 inhibitor administered according to the provided methods is at least one of: (1H-Benzo[g]indol-2-yl)-phenyl-methanone; (3H-Benzo[e]indol-2-yl)-phenyl-methanone; (3H-Benzo[e]indol-2-yl)-(4-methoxy-phenyl)-methanone; (3H-Benzo[e]indol-2-yl)-pyridin-4-yl-methanone; HCl salt of (3H-Benzo[e]indol-2-yl)-pyridin-4-yl-methanone; (3H-Benzo[e]indol-2-yl)-(3-methoxy-phenyl)-methanone; (3H-Benzo[e]indol-2-yl)-pyridin-3-yl-methanone; (3H-Benzo[e]indol-2-yl)-(2-methoxy-phenyl)-methanone; (3H-Benzo[e]indol-2-yl)-(2-hydroxy-phenyl)-methanone; (3H-Benzo[e]indol-2-yl)-(4-hydroxy-phenyl)-methanone; (5-Methyl-3H-benzo[e]indol-2-yl)-phenyl-methanone; Phenyl-(7H-pyrrolo[2,3-h]quinolin-8-yl)-methanone; (3H-Benzo[e]indol-2-yl)-(3-hydroxy-phenyl)-methanone; (3H-benzo[e]indol-2-yl)-(2-chloro-pyridin-4-yl)-methanone; (3H-benzo[e]indol-2-yl)-(1-oxy-pyridin-4-yl)-methanone; Phenyl-(6,7,8,9-tetrahydro-3H-benzo[e]indol-2-yl)-methanone; (3H-Benzo[e]indol-2-yl)-(4-hydroxy-3-methoxylthenyl)-methanone; (3H-Benzo[e]indol-2-yl)-(4-benzyloxy-3-methoxy-phenyl)-methanone; 4-(3H-Benzo[e]indole-2-carbonyl)-benzoic acid methyl ester; 4-(3H-Benzo[e]indole-2-carbonyl)-benzoic acid; (4-Amino-phenyl)-(3H-benzo[e]indol-2-yl)-methanone; 5-(3H-Benzo[e]indole-2-carbonyl)-2-benzyloxy-benzoic acid methyl; 5-(3H-Benzo[e]indole-2-carbonyl)-2-benzyloxy-benzoic Acidmethanone; (3H-Benzo[e]indol-2-yl)-(2-methoxy-pyridin-4-yl)-methanone; (5-Fluoro-3H-benzo[e]indol-2-yl)-(3-methoxy-phenyl)-methanone; (5-Fluoro-3H-benzo[e]indol-2-yl)-pyridin-4-yl-methanone; (4-Benzyloxy-3-methoxy-phenyl)-(5-fluoro-3H-benzo[e]indol-2-yl)-methanone; (5-Fluoro-3H-benzo[e]indol-2-yl)-(4-hydroxy-3-methoxy-phenyl)-methanone; (3H-Benzo[e]indol-2-yl)-(3-hydroxymethyl-phenyl)-methanone; Cyclohexyl-(5-fluoro-3H-benzo[e]indol-2-yl)-methanone; (5-Fluoro-3H-benzo[e]indol-2-yl)-(3-fluoro-4-hydroxy-phenyl)-methanone; (3H-Benzo[e]indol-2-yl)-p-tolyl-methanone; (3H-Benzo[e]indol-2-yl)-(3-methoxy-phenyl-methanol; (3H-Benzo[e]indol-2-yl)-pyridin-4-yl-methanol; 3H-Benzo[e]indole-2-carboxylic acid phenylamide; 3H-Benzo[e]indole-2-carboxylic acid (3-methoxy-phenyl)-amide; (3H-Benzo[e]indol-2-yl)-(4-dimethylamino-phenyl)-methanone; (4-Amino-3-methoxy-phenyl)-(3H-benzo[e]indol-2-yl)-methanone; (4-Amino-3-methoxy-phenyl)-(5-hydroxy-3H-benzo[e]indol-2-yl)-methanone; (4-Amino-3-methoxy-phenyl)-(5-methoxy-3H-benzo[e]indol-2-yl)-methanone; N-[4-(3H-Benzo[e]indole-2-carbonyl)-phenyl]-methanesulfonamide; 3H-Benzo[e]indole-2-carboxylic acid (4-amino-phenyl)-amide; (4-Amino-phenyl)-(5-methoxy-3H-benzo[e]indol-2-yl)-methanone; (4-Amino-2-fluoro-phenyl)-(5-methoxy-3H-benzo[e]indol-2-yl)-methanone; (4-Amino-3-fluoro-phenyl)-(5-methoxy-3H-benzo[e]indol-2-yl)-methanone; (4-Amino-2-methoxy-phenyl)-(5-methoxy-3H-benzo[e]indol-2-yl)-methanone; (4-Amino-phenyl)-(9-methoxy-3H-benzo[e]indol-2-yl)-methanone; (4-Amino-3-methoxy-phenyl)-(9-methoxy-3H-benzo[e]indol-2-yl)-methanone; (4-Amino-2-methoxy-phenyl)-(9-methoxy-3H-benzo[e]indol-2-yl)-methanone; (4-Amino-3-fluoro-phenyl)-(9-methoxy-3H-benzo[e]indol-2-yl)-methanone; (4-Amino-2-fluoro-phenyl)-(9-methoxy-3H-benzo[e]indol-2-yl)-methanone; (4-Amino-3-fluoro-phenyl)-(3H-benzo[e]indol-2-yl)-methanone; (4-Amino-2-fluoro-phenyl)-(3H-benzo[e]indol-2-yl)-methanone; (4-Amino-phenyl)-(7-methoxy-3H-benzo[e]indol-2-yl)-methanone; (4-Amino-phenyl)-(5-hydroxy-3-methyl-3H-benzo[e]indol-2-yl)-methanone; (7-Amino-5-fluoro-9-hydroxy-3H-benzo[e]indol-2-yl)-(3-methyl-pyridin-4-yl)-methanone; (5-Amino-3H-pyrrolo[3,2-f]isoquinolin-2-yl)-(3-methoxy-pyridin-4-yl)-methanone; (4-Amino-2-methyl-phenyl)-(9-hydroxy-3H-pyrrolo[2,3-c]quinolin-2-yl)-methanone; and (4-Amino-phenyl)-(7-methanesulfonyl-3H-benzo[e]indol-2-yl)-methanone, or a salt thereof.

In some embodiments, the PFKFB3 inhibitor administered according to the provided methods is at least one of: 1-Pyridin-4-yl-3-quinolin-4-yl-propenone; 1-Pyridin-4-yl-3-quinolin-3-yl-propenone; 1-Pyridin-3-yl-3-quinolin-2-yl-propenone; 1-Pyridin-3-yl-3-quinolin-4-yl-propenone; 1-Pyridin-3-yl-3-quinolin-3-yl-propenone; 1-Naphthalen-2-yl-3-quinolin-2-yl-propenone; 1-Naphthalen-2-yl-3-quinolin-3-yl-propenone; 1-Pyridin-4-yl-3-quinolin-3-yl-propenone; 3-(4-Hydroxy-quinolin-2-yl)-1-pyridin-4-yl-propenone; 3-(8-Hydroxy-quinolin-2-yl)-1-pyridin-3-yl-propenone; 3-Quinolin-2-yl-1-p-tolyl-propenone; 3-(8-Hydroxy-quinolin-2-yl)-1-pyridin-4-yl-propenone; 3-(8-Hydroxy-quinolin-2-yl)-1-p-tolyl-propenone; 3-(4-Hydroxy-quinolin-2-yl)-1-p-tolyl-propenone; 1-Phenyl-3-quinolin-2-yl-propenone; 1-Pyridin-2-yl-3-quinolin-2-yl-propenone; 1-(2-Hydroxy-phenyl)-3-quinolin-2-yl-propenone; 1-(4-Hydroxy-phenyl)-3-quinolin-2-yl-propenone; 1-(2-Amino-phenyl)-3-quinolin-2-yl-propenone; 1-(4-Amino-phenyl)-3-quinolin-2-yl-propenone; or a salt thereof.

In some embodiments, the PFKFB3 inhibitor administered according to the provided methods is at least one of: 4-(3-Quinolin-2-yl-acryloyl)-benzamide; 4-(3-Quinolin-2-yl-acryloyl)-benzoic acid; 3-(8-Methyl-quinolin-2-yl)-1-pyridin-4-yl-propenone; 1-(2-Fluoro-pyridin-4-yl)-3-quinolin-2-yl-propenone; 3-(8-Fluoro-quinolin-2-yl)-1-pyridin-4-yl-propenone; 3-(6-Hydroxy-quinolin-2-yl)-1-pyridin-4-yl-propenone; 3-(8-Methylamino-quinolin-2-yl)-1-pyridin-4-yl-propenone; 3-(7-Methyl-quinolin-2-yl)-1-pyridin-4-yl-propenone; and 1-Methyl-4-[3-(8-methyl-quinolin-2-yl)-acryloyl]-pyridinium, or a salt thereof.

In some embodiments, the PFKFB3 inhibitor administered according to the provided methods is at least one of: PFK15 (1-(4-pyridinyl)-3-(2-quinolinyl)-2-propen-1-one); (2S)—N-[4-[[3-Cyano-1-(2-methyl-propyl)-1H-indol-5-yl]oxy]phenyl]-2-pyrrolidine-carboxamide 3PO (3-(3-Pyridinyl)-1-(4-pyridinyl)-2-propen-1-one); (2S)—N-[4-[[3-Cyano-1-[(3,5-dimethyl-4-isoxazolyl)methyl]-1H-indol-5-yl]oxy]phenyl]-2-pyrrolidine-carboxamide; and Ethyl 7-hydroxy-2-oxo-2H-1-benzopyran-3-carboxylate, or a salt thereof.

In a particular embodiment, the PFKFB3 inhibitor administered according to the provided methods is PFK15, or a salt thereof.

In a particular embodiment, the PFKFB3 inhibitor administered according to the provided methods is PFK158 ((E)-1-(4-Pyridinyl)-3-[7-(trifluoromethyl)-2-quinolinyl]-2-propen-1-one), or a salt thereof.

In a particular embodiment, the PFKFB3 inhibitor administered according to the provided methods is BrAcNHEtOP (N-bromoacetylethanolamine phosphate), or a salt thereof.

In a particular embodiment, the PFKFB3 inhibitor administered according to the provided methods is AZ67, or a salt thereof.

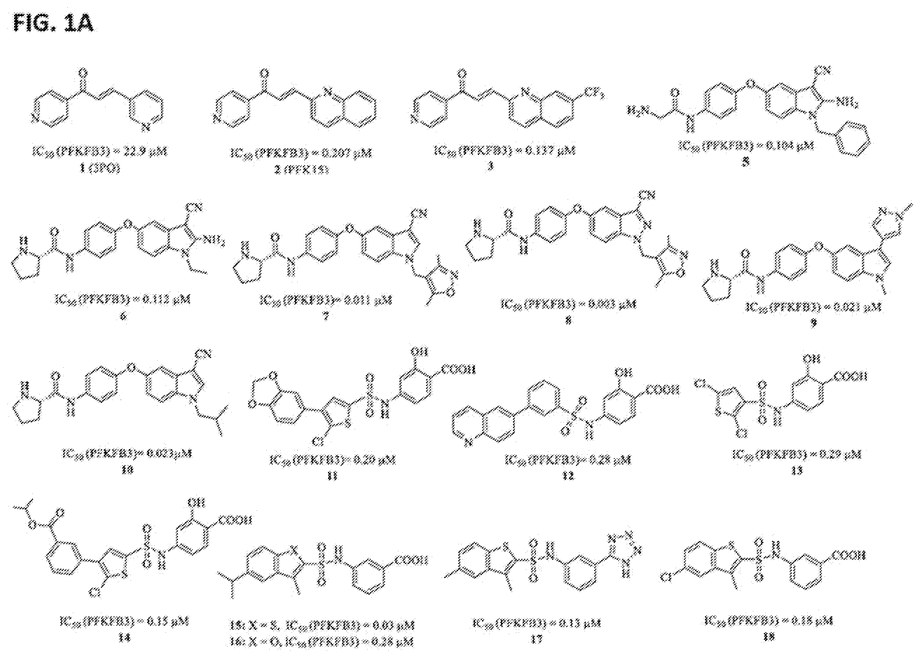

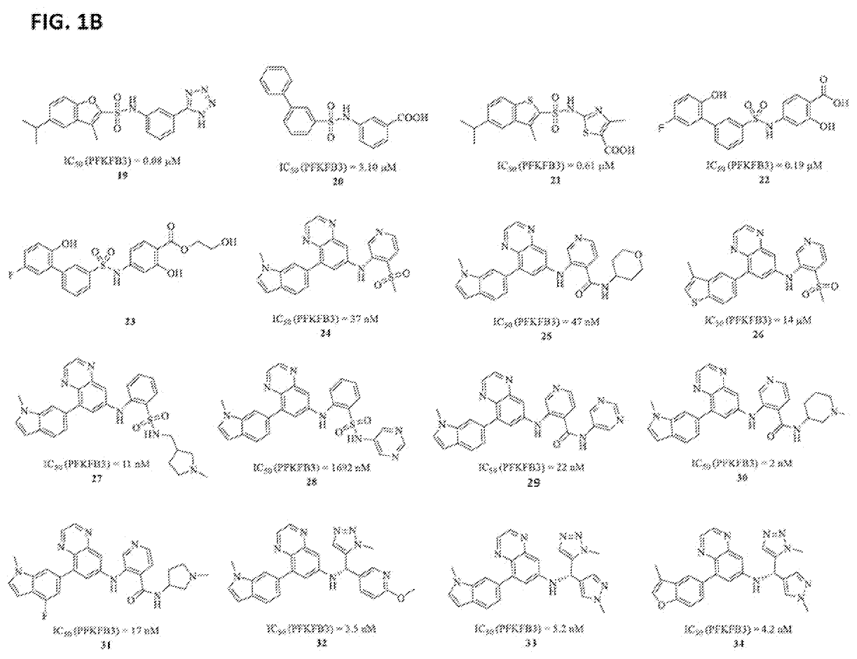

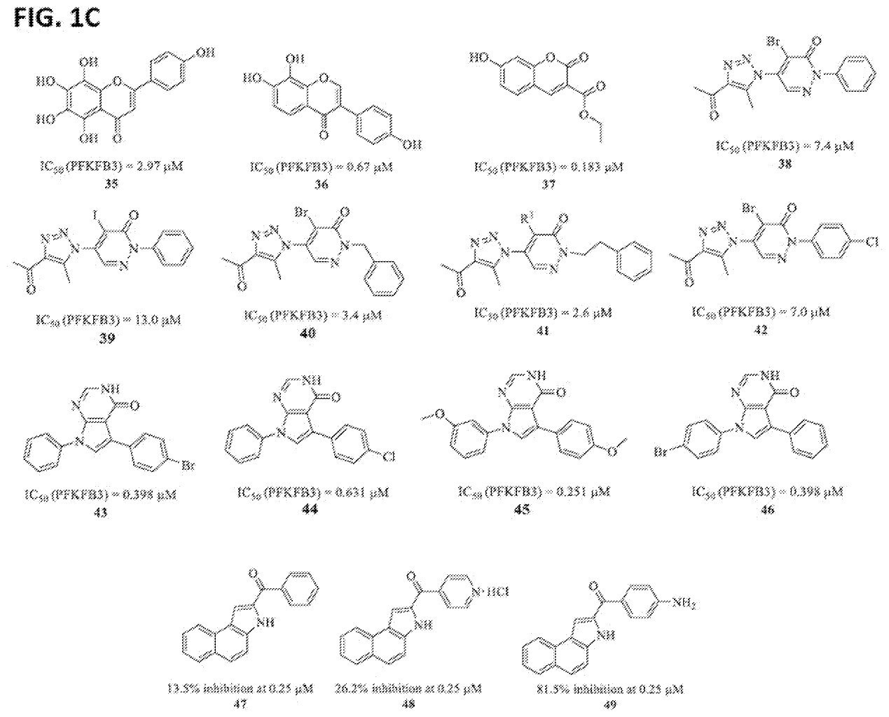

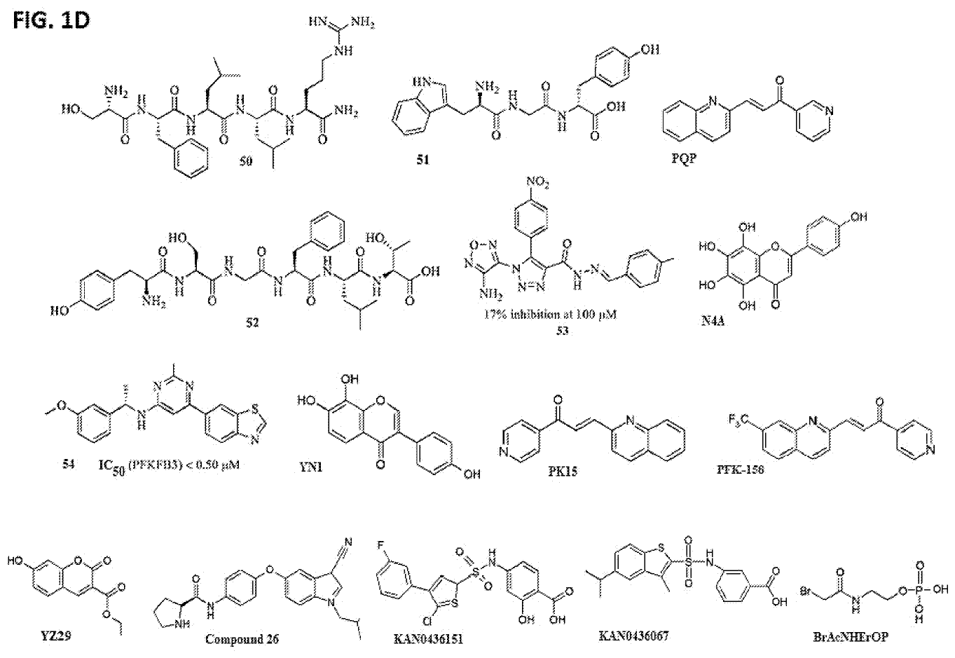

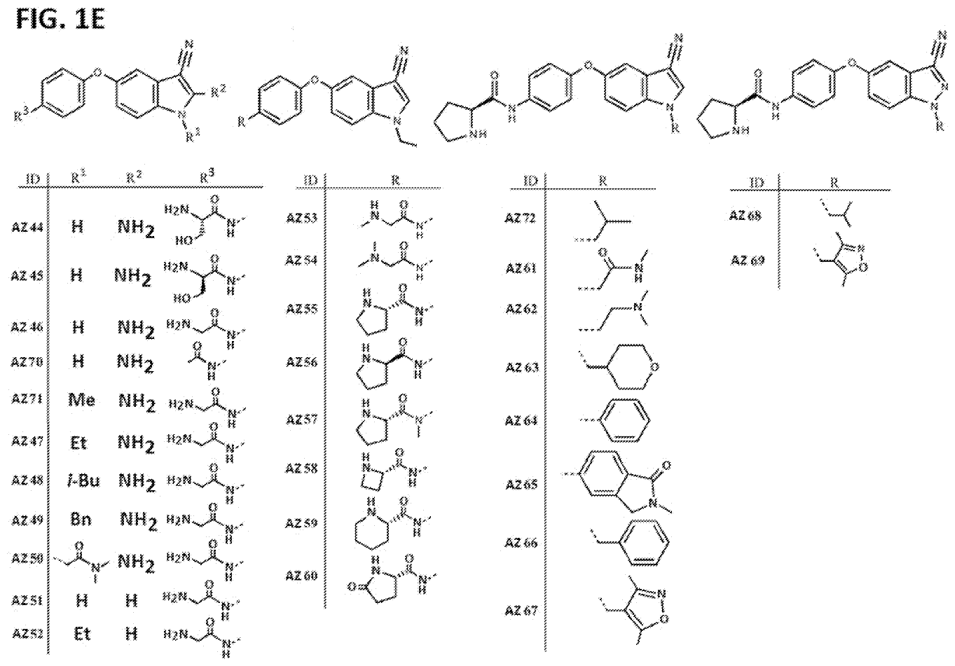

In some embodiments, the PFKFB3 inhibitor administered according to the provided methods is at least one PFKFB3 inhibitor having the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In other embodiments, the PFKFB3 inhibitor administered according to the provided methods is the PFKFB3 inhibitor having the structure of formula AZ44-AZ70 or AZ71, depicted in FIG. 1E, or a salt thereof.

In a particular embodiment, the PFKFB3 inhibitor administered according to the provided methods is KAN0436151, or a salt thereof.

In a particular embodiment, the PFKFB3 inhibitor administered according to the provided methods is KAN0436067, or a salt thereof.

### HIF1-α Pathway Inhibitors

Hypoxia-inducible factor 1-alpha (HIF-1-alpha) is a subunit of a heterodimeric transcription factor hypoxia-inducible factor 1 (HIF-1) that is considered to be the master transcriptional regulator of cellular and developmental response to hypoxia.

In some embodiments, the disclosure provides a method of treating a cardiovascular disease in a subject in need thereof that comprises:
(a) administering an effective amount of a HIF1-α Pathway Inhibitor or a HIF1-α Inhibitor and an PFKFB3 inhibitor to the subject;
(b) administering an effective amount of a HIF1-α Pathway Inhibitor or a HIF1-α Inhibitor to the subject, wherein the subject has previously been administered a PFKFB3 Inhibitor; or
(c) administering an effective amount of a PFKFB3 Inhibitor to the subject wherein the subject has previously been administered a HIF1-α Pathway Inhibitor or a HIF1-α Inhibitor; and
wherein the PFKFB3 inhibitor does not inhibit PI3K/AKT/mTOR pathway or HIF1-α.

The term “HIF1-α Pathway-α Inhibitor” as used herein refers to a composition that inhibits or reduces HIF1-α directly or indirectly via inhibiting one or more activities of the PI3K/AKT/mTOR pathway that is upstream of the HIF1-α pathway. The term “HIF1-α Inhibitor” is used herein to refer to a composition that inhibits or reduces HIF1-α directly. Thus, for example, mTOR pathway inhibitors such as temsirolimus, everolimus, and sirolimus are considered herein to be “HIF1-α Pathway-α Inhibitors”, but not “HIF1-α Inhibitors.”

The “HIF1-α Pathway-α Inhibitors” that can be administered according to the provided methods are not particularly limited. In some embodiments, the administered HIF1-α Pathway Inhibitor is an antibody or a HIF1-α-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, ENMD-1198, miRNA, dsRNA, ssRNA, and shRNA), a peptibody, a nanobody, a HIF1-α Pathway binding polypeptide, or a small molecule HIF1-α Pathway Inhibitor

In some embodiments, the administered HIF1-α Pathway Inhibitor administered according to the provided methods has an IC50 for a HIF1-α activity/function of 100 μM or lower concentration for a HIF1-α activity. In some embodiments, the HIF1-α Pathway Inhibitor has an IC50 of at least or at most or about 200, 100, 80, 50, 40, 20, 10, 5, or 1 μM, or at least or at most or about 100, 10, or 1 nM, or lower (or any range or value derivable therefrom). In some embodiments, the HIF1-α Pathway Inhibitor inhibits the expression of HIF1-α. Assays for determining the ability of a compound to inhibit HIF1-α activity are known in the art. In some embodiments, the inhibition of HIF1-α activity or expression is a decrease as compared with a control level or sample. In some embodiments, a functional assay such as an MTT assay, cell proliferation assay, BRDU or Ki67 immunofluorescence assay, apoptosis assay, or glycolysis assay is used to assay for the ability of a composition to inhibit HIF1-α activity.

The HIF1-α Inhibitors that can be administered according to the provided methods are not particularly limited. In some embodiments, the HIF1-α Inhibitor modulates one or more of HIF-1α mRNA expression; HIF-1α protein translation or degradation; HIF-1α/HIF-1β dimerization; HIF-1α-DNA binding (e.g., HIF-1α/HRE); and/or HIF-1α transcriptional activity (e.g., CH-1 of p300/C-TAD of HIF-1α).

In some embodiments, the HIF1-α Inhibitor administered according to the provided methods is a small molecule. In some embodiments, the HIF1-α Inhibitor administered according to the provided methods is a protein or polypeptide (e.g., an anti HIF1 antibody or antibody fragment that binds HIF1). In some embodiments, the HIF1-α Inhibitor administered according to the provided methods is a therapeutic nucleic acid (e.g., an aptamer, antisense molecule, ribozyme, a Dicer substrate, siRNA, miRNA, dsRNA, ssRNA, or an shRNA).

In some embodiments, the HIF1-α Pathway Inhibitor administered according to the provided methods is a HIF1-α Pathway Inhibitor (e.g., a PI3K pathway inhibitor, a MAPK pathway inhibitor, an Akt pathway inhibitor, and/or an mTOR inhibitor); a HIF translation inhibitor (e.g., a topoisomerase inhibitor, a microtubule targeting drug a cardiac glycoside, or an antisense HIF-1α mRNA); an inhibitor of HIF stability, nuclear localization or dimerization (e.g., acriflavine or an HDAC inhibitor); an inhibitor of HIF transactivation (e.g., a HIF1 coactivator recruitment inhibitor or a HIF1 DNA binding inhibitor).

In some embodiments, the HIF1-α Inhibitor administered according to the provided methods is a HIF1-α Pathway Inhibitor (e.g., a PI3K pathway inhibitor, a MAPK pathway inhibitor, an Akt pathway inhibitor, and/or an mTOR inhibitor). In some embodiments, the HIF1-α Inhibitor administered according to the provided methods is a PI3K pathway inhibitor. In one embodiment, the administered HIF1-α Pathway Inhibitor is P3155, LY29, LY294002, wortmannin, or GDC-0941. In one embodiment, the administered HIF1-α Pathway Inhibitor is resveratrol. In another embodiment, the administered HIF1-α Pathway Inhibitor is a glyceolin. In some embodiments, the HIF1-α Pathway Inhibitor administered according to the provided methods is an mTOR inhibitor. In one embodiment, the administered HIF1-α Pathway Inhibitor is rapamycin, temsirolimus (CC1-779), everolimus, sirolimus, or PP242.

In a particular embodiment, the administered HIF1-α Inhibitor is silibinin.

In some embodiments, the HIF1-α Inhibitor administered according to the provided methods is a HIF translation inhibitor. In one embodiment, the administered HIF1-α Inhibitor is PX-478 (S-2-amino-3-[4′-N,N-bis(chloroethyl)[amino]phenyl propionic acid N-oxide dihydrochloride), NSC-64421, camptothecin (CPT), SN38, irinotecan, topotecan, NSC-644221, cycloheximide, or apigenin, or a salt thereof. In one embodiment, the administered HIF1-α Inhibitor is aminoflavone, KC7F2 (N,N′-(disulfanediylbis(ethane-2,1-diyl))bis(2,5-dichlorobenzene-sulfonamide), 2-meth-oxyestra-diol (2ME2) or an analog or salt thereof. In one embodiment, the administered HIF1-α Inhibitor is ENMD-1198, ENMD-1200, or ENMD-1237, or a salt thereof. In one embodiment, the administered HIF1-α Inhibitor is EZN-2208, or a salt thereof. In one embodiment, the administered HIF1-α Inhibitor is EZN-2968, or a salt thereof.

In a particular embodiment, the administered HIF1-α Inhibitor is PX-478, or a salt thereof.

In some embodiments, the HIF1-α Inhibitor administered according to the provided methods is a cardiac glycoside. In one embodiment, the administered cardiac glycoside is digoxin, or a salt thereof. In another embodiment, the administered cardiac glycoside ouabain or proscillardin A, or a salt thereof.

In some embodiments, the HIF1-α Pathway Inhibitor administered according to the provided methods is a topoisomerase inhibitor. In one embodiment, the administered topoisomerase inhibitor is camptothecin (CPT), SN38, irinotecan, or topotecan (e.g., PEG-SN38), or a salt thereof.

In some embodiments, the HIF1-α Pathway Inhibitor administered according to the provided methods is a microtubule targeting drug. In one embodiment, the administered microtubule targeting drug is 2 methoxyestradiol (2ME2), ENMD-1198, ENMD-1200, ENMD-1237, or Taxotere, or a salt thereof.

In some embodiments, the HIF1-α Inhibitor administered according to the provided methods is a therapeutic nucleic acid. In some embodiments therapeutic nucleic acid is an aptamer, antisense molecule, ribozyme, a Dicer substrate, siRNA, miRNA, dsRNA, ssRNA, and shRNA). In some embodiments, therapeutic nucleic acid is an antisense oligonucleotide.

In some embodiments, the HIF1-A Inhibitor administered according to the provided methods is a siRNA or an antisense oligonucleotide. In one embodiment, the administered HIF1-α Inhibitor is EZN-2968. In one embodiment, the administered HIF1-α Inhibitor is RX-0047.

Representative examples of human HIF1-A coding sequences are provided in GenBank accession numbers NM_004566.3, NM_001145443.2, NP_001138915.1, NM_001282630.2, NM_001314063.1, NM_001323016.1, NM_001323017.1, and NM_001363545.2. The sequences associated with the each of these Genbank accession numbers is hereby incorporated by reference herein in its entirety for all purposes. Therapeutic nucleic acids that inhibit HIF1-A activity can routinely be designed and prepared based on each of the above human HIF1-A transcript sequences using methods known in the art.

The administration of HIF1-A inhibitory nucleic acids or any ways of inhibiting gene expression of HIF1-A known in the art are contemplated in certain embodiments of the provided methods. Examples of inhibitory (therapeutic) nucleic acid include but are not limited to, antisense nucleic acids such as: a small interfering RNA (siRNA), short hairpin RNA (shRNA), double-stranded RNA, and any other antisense oligonucleotide. Also included are ribozymes or nucleic acids encoding any of the inhibitors described herein. An inhibitory nucleic acid may inhibit the transcription of HIF1-A or prevent the translation of a HIF1-A gene transcript in a cell. In some embodiments, the HIF1-A inhibitory nucleic acid administered according to the provided methods is from 16 to 1000 nucleotides in length. In certain embodiments the administered HIF1-A inhibitory nucleic acid is from 18 to 100 nucleotides long. In certain embodiments the administered HIF1-A inhibitory nucleic acid at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 nucleotides or any range derivable therefrom.

In some embodiments, the HIF1-A inhibitory nucleic acid administered according to the provided methods is capable of decreasing the expression of HIF1-A by at least 10%. 20%, 30%, or 40%, more particularly by at least 50%, 60%, or 70%, and most particularly by at least 75%, 80%, 90%, 95% or more or any range or value in between the foregoing.

In some embodiments, the HIF1-A inhibitory nucleic acid administered according to the provided methods is between 17 to 25 nucleotides in length and comprises a 5′ to 3′ sequence that is at least 90% complementary to the 5′ to 3′ sequence of a mature HIF1-A mRNA (e.g., as disclosed in any one or more of GenBank accession nos. NM_001530.4, NM_181054.3, and NM_001243084.2). In some embodiments, the administered HIF1-A inhibitory nucleic acid is 17, 18, 19, 20, 21, 22, 23, 24, or 25 nucleotides in length, or any range derivable therein. In some embodiments, the administered HIF1-A inhibitory nucleic acid has a sequence (from 5′ to 3′) that is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the corresponding 5′ to 3′ sequence of a mature HIF1-A mRNA (e.g., as disclosed in any one or more of GenBank accession nos. NM_001530.4, NM_181054.3, and NM_001243084.2). One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the HIF1-A inhibitory nucleic acid administered according to the provided methods is a miRNA mimic. In some embodiments, the administered HIF1-α Inhibitor is a miR-483 mimic.

In some embodiments, the HIF1-α Inhibitor administered according to the provided methods is an inhibitor of HIF stability, nuclear localization or dimerization. In one embodiment, the inhibitor administered according to the provided methods destabilizes HIF. In one embodiment, the inhibitor administered according to the provided methods is a histone deacetylase inhibitor (HDACI). In a further embodiment, the administered HDACI is LW6/CAY10585, vorinostat, romidepsin (FK228), panobinostat, belinostat, Trichostatin A (TSA), LAQ824, or phenethyl isothiocyanate, or a salt thereof. In one embodiment, the inhibitor administered according to the provided methods is PX-12/pleurotin, HIF-1α inhibitor (CAS No. 934593-90-5), cryptotanshinone, or BAY 87-2243 (1-cyclopropyl-4-[4-[[5-methyl-3-[3-[4-(trifluoromethoxy)phenyl]-1,2,4-oxadiazol-5-yl]-1H-pyrazol-1-yl]methyl]-2-pyridinyl]-piperazine), or a salt thereof. In one embodiment, the inhibitor administered according to the provided methods is IDF-11774, Bisphenol A/Dimethyl bisphenol A, or a salt thereof. Chrysin (5,7-dihydroxy-flavone), or SCH66336, or a salt thereof. In one embodiment, the inhibitor administered according to the provided methods is geldanamycin or analog thereof, 17-AAG (tanespimycin: allylamino-17-demethoxygeldanamycin), 17-DMAG (alvespimycin), 17AG, radiccicol, KF58333, ENMD-1198, ENMD-1237, or ganetasipib, or a salt thereof. In one embodiment, the inhibitor administered according to the provided methods interferes with HIF-dimerization. In one embodiment, the inhibitor administered according to the provided methods is acriflavine, or a salt thereof. In one embodiment, the inhibitor administered according to the provided methods is TC-S7009, PT2385, or TAT-cyclo-CLLFVY, or a salt thereof.

In a particular embodiment, the inhibitor administered according to the provided methods is ganetasipib, or a salt thereof.

In a particular embodiment, the inhibitor administered according to the provided methods is BAY 87-2243.

In some embodiments, the HIF1-α Pathway Inhibitor administered according to the provided methods is a histone deacetylase inhibitor (HDACI). In one embodiment, the administered HDACI is LW6/CAY10585 (methyl 3-(2-(4-(adamantan-1-yl)phenoxy) acetamido)-4-hydroxy-benzoate, vorinostat, romidepsin (FK228), panobinostat, belinostat, Trichostatin A (TSA), LAQ824, or phenethyl isothiocyanate, or a salt thereof.

In some embodiments, the HIF1-α Pathway Inhibitor administered according to the provided methods is a heat shock protein inhibitor. In one embodiment, the administered HIF1-α Pathway Inhibitor is an HSP90 inhibitor. In one embodiment, the administered HSP90 inhibitor is a geldanamycin or analog thereof, 17-AAG (tanespimycin: allylamino-17-demethoxy geldanamycin), 17-DMAG (alvespimycin), 17AG, radiccicol, KF58333, ENMD-1198, ENMD-1237, or ganetasipib, or a salt thereof. In a particular embodiment, the administered heat shock protein inhibitor is ganetasipib, or a salt thereof. In one embodiment, the administered HIF1-α Pathway Inhibitor is an HSP70 inhibitor. In one embodiment, the administered HSP70 inhibitor is triptolide, or a salt thereof.

In some embodiments, the HIF1-α Inhibitor administered according to the provided methods is an inhibitor of HIF transactivation. In one embodiment, the HIF1-α Inhibitor administered according to the provided methods inhibits HIF coactivator recruitment. In one embodiment, the administered HIF1-α Inhibitor is chetomin, YC-1, or KCN-1 (3,4-dimethoxy-N-[(2,2-dimethyl-2H-chromen-6-yl)methyl]-N-phenylbenzenesulfonamide), or a salt thereof. In another particular embodiment, the administered HIF1-α Inhibitor is NSC 607097, or a salt thereof. In one embodiment, the administered HIF1-α Inhibitor is a proteasome inhibitor. In a further embodiment, the administered inhibitor is bortezomib or carfilzomib, or a salt thereof. In one embodiment, the administered HIF1-α Inhibitor is indenopyrazole 21, FM19G11, flavopiridol, Amphotericin B, actinomycin, AJM290, or AW464, or a salt thereof. In one embodiment, the administered HIF1-α Inhibitor is triptolide, or a salt thereof.

In a particular embodiment, the HIF1-α Inhibitor administered according to the provided methods is YC-1, or a salt thereof.

In some embodiments, the HIF1-α Inhibitor administered according to the provided methods is an antibody that binds HIF1-α or a HIF1-α-binding antibody fragment (e.g., a single chain antibody, a single-domain antibody (e.g., the AG1-5 VHH), a Fab fragment, F(ab′)2 fragment, Fd fragment; Fv fragment, scFv, dAb fragment, or another engineered molecule, such as a diabody, triabody, tetrabody, minibody, and a minimal recognition unit). In a particular embodiment, the administered HIF1-α Inhibitor is a VHH or nanobody. In one embodiment, the administered antibody is AGI-5. In one embodiment, the administered antibody is AHPC.

In some embodiments, the HIF1-α Inhibitor administered according to the provided methods is an inhibitor of HIF1 DNA-binding. In one embodiment, the administered HIF1-α Inhibitor is echinomycin (NSC-13502) or Compound DJ12.162. In one embodiment, the administered HIF1-α Inhibitor is an anthracycline. In a further embodiment, the administered inhibitor is doxorubicin or danuorubicin. In one embodiment, the administered HIF1-α Inhibitor is a polyamide. In some embodiments, the HIF1-α Inhibitor is an antibody that binds HIF1-α or is a HIF1-α-binding antibody fragment such as a VHH or nanobody.

In some embodiments, the HIF1-A Inhibitor administered according to the provided methods is a therapeutic nucleic acid. In some embodiments the therapeutic nucleic acid is an aptamer, antisense molecule, ribozyme, a Dicer substrate, ENMD-1198, miRNA, dsRNA, ssRNA, and shRNA). In some embodiments, the therapeutic nucleic acid is ENMD-1198 or an antisense oligonucleotide.

In some embodiments, the HIF1-A Inhibitor administered according to the provided methods is an siRNA or an antisense oligonucleotide. In some embodiments, the administered HIF1-A Inhibitor inhibitor is RX-0047. In some embodiments, the administered HIF1-A Inhibitor inhibitor is EZN-2968.

Representative examples of human HIF1-A coding sequences are provided in GenBank accession numbers NM_004566.3, NM_001145443.2, NP_001138915.1, NM_001282630.2, NM_001314063.1. NM_001323016.1. NM_001323017.1, and NM_001363545.2. The sequences associated with the each of these Genbank accession numbers is hereby incorporated by reference herein in its entirety for all purposes. Therapeutic nucleic acids that inhibit HIF1-A activity can routinely be designed and prepared based on each of the above human HIF1-A transcript sequences using methods known in the art.

The administration of HIF1-A inhibitory nucleic acids or any ways of inhibiting gene expression of HIF1-A known in the art are contemplated in certain embodiments of the provided methods. Examples of inhibitory nucleics acid include but are not limited to, antisense nucleic acids such as: a small interfering RNA (siRNA), short hairpin RNA (shRNA), double-stranded RNA, and any other antisense oligonucleotide. Also included are ribozymes or nucleic acids encoding any of the inhibitors described herein. An inhibitory nucleic acid may inhibit the transcription of HIF1-A or prevent the translation of a HIF1-A gene transcript in a cell. In some embodiments, the HIF1-A inhibitory nucleic acid adminstered according to the provided methods is from 16 to 1000 nucleotides in length. In certain embodiments the administered HIF1-A inhibitory nucleic acid is from 18 to 100 nucleotides long. In certain embodiments the administered HIF1-A inhibitory nucleic acid at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 nucleotides or any range derivable therefrom.

In some embodiments, the HIF1-A inhibitory nucleic acid adminstered according to the provided methods is capable of decreasing the expression of HIF1-A by at least 10%. 20%, 30%, or 40%, more particularly by at least 50%, 60%, or 70%, and most particularly by at least 75%, 80%, 90%, 95% or more or any range or value in between the foregoing.

In some embodiments, the HIF1-A inhibitory nucleic acid adminstered according to the provided methods is between 17 to 25 nucleotides in length and comprises a 5′ to 3′ sequence that is at least 90% complementary to the 5′ to 3′ sequence of a mature HIF1-A mRNA (e.g., as disclosed in any one or more of GenBank accession nos. NM_001530.4, NM_181054.3, and NM_001243084.2). In some embodiments, the administered HIF1-A inhibitory nucleic acid is 17, 18, 19, 20, 21, 22, 23, 24, or 25 nucleotides in length, or any range derivable therein. In some embodiments, the administered HIF1-A inhibitory nucleic acid has a sequence (from 5′ to 3′) that is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the corresponding 5′ to 3′ sequence of a mature HIF1-A mRNA (e.g., as disclosed in any one or more of GenBank accession nos. NM_001530.4. NM_181054.3, and NM_001243084.2). One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the HIF1-A inhibitory nucleic acid administered according to the provided methods is a miRNA mimic. In some embodiments, the administered HIF1-α Inhibitor is a miR-483 mimic.

In some embodiments, the HIF1-α Inhibitor administered according to the provided methods is a therapeutic nucleic acid. In some embodiments the therapeutic nucleic acid is an ENMD-1198 molecule or antisense oligonucleotide.

### Kits for Administration of Active Agents

In another embodiment, the disclosure provides a kit containing a HIF1-α Pathway Inhibitor and a PFKFB3 inhibitor and/or other therapeutic and delivery agents. In some embodiments, a kit for preparing and/or administering a therapy described herein may be provided. The kit may comprise one or more sealed vials containing any of the pharmaceutical compositions, therapeutic agents and/or other therapeutic and delivery agents. In some embodiments, the kits comprise lipid delivery systems. In some embodiments, the lipid is in one vial, and the therapeutic agent is in a separate vial. The kit may include, for example, at least one inhibitor of PFKFB3 expression/activity, at least one inhibitor of HIF1-alpha expression/activity, and one or more reagents to prepare, formulate, and/or administer the components described herein or perform one or more steps of the methods. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill. The instruction information may be in a computer readable media containing machine-readable instructions that, when executed using a computer, cause the display of a real or virtual procedure of delivering a pharmaceutically effective amount of a therapeutic agent.

In some embodiments, kits may be provided to evaluate the expression of PFKFB3 and/or HIF-α or related molecules. Such kits can be prepared from readily available materials and reagents. For example, such kits can comprise any one or more of the following materials: enzymes, reaction tubes, buffers, detergent, primers and probes, nucleic acid amplification, and/or hybridization agents. In a particular embodiment, these kits allow a practitioner to obtain samples in blood, tears, semen, saliva, urine, tissue, serum, stool, colon, rectum, sputum, cerebrospinal fluid and supernatant from cell lysate. In another embodiment, these kits include the needed apparatus for performing RNA extraction, RT-PCR, and gel electrophoresis. Instructions for performing the assays can also be included in the kits.

Kits may comprise components, which may be individually packaged or placed in a container, such as a tube, bottle, vial, syringe, or other suitable container means. The components may include probes, primers, antibodies, arrays, negative and/or positive controls. Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

The kit can further comprise reagents for labeling PFKFB3 and/or HIF-1 alpha in the sample. The kit may also include labeling reagents, including at least one of amine-modified nucleotide, poly(A) polymerase, and poly(A) polymerase buffer. Labeling reagents can include an amine-reactive dye or any dye known in the art.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits may also include a means for containing the nucleic acids, antibodies or any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. Alternatively, the components of the kit may be provided as dried powder(s). When reagents and/or components are provided as a dry powder, the powder can be reconstituted by the addition of a suitable solvent. It is envisioned that the solvent may also be provided in another container means. The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits may include a means for containing the vials in close confinement for commercial sale, such as, e.g., injection and/or blow-molded plastic containers into which the desired vials are retained. The kit may also include instructions for employing the kit components as well the use of any other reagent not included in the kit. Instructions may include variations that can be implemented.

### Methods of Administration

The regimen of administration according to a method provided herein (e.g., dose combined with frequency of administration) will generally involve administration in an amount and at a frequency to provide for a desired effect, e.g., administration of an amount effective to provide for improvement in one or more symptoms of a cardiovascular disease in a subject such as one or more symptoms associated with AD, or neural injury. Administration of each drug in combination may be by any suitable means that can be combined with other ingredients to alleviate the condition of the patient or cause concentration of the drug to effectively treat the disease or disorder. Possible compositions include those suitable for oral, rectal, topical (including transdermal, oral and sublingual), or parenteral (including subcutaneous, intramuscular, intravenous and intradermal) administration.

In some embodiments of the present invention, compositions are administered to a patient alone or in combination with other therapies, pharmaceuticals, supplements, and/or a specified diet, or in pharmaceutical compositions where it is mixed with excipient(s) or other pharmaceutically acceptable carriers. Depending on the goal of administration (e.g., severity of condition, duration of treatment, etc.), compositions (e.g., comprising a compound of Formula I, such as DMB) may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration. In some embodiments, a compound of Formula I (e.g., DMB) may be administered in the form of a solid, semi-solid or liquid dosage form: such as tablet, capsule, pill, powder, suppository, solution, elixir, syrup, suspension, cream, lozenge, paste and spray formulated appropriately to provide the desired therapeutic profile. As those skilled in the art would recognize, depending on the chosen route of administration, the composition form is selected.

The phrases “parenteral administration” and “administered parenterally” as used herein refer to modes of administration other than enteral and topical administration, such as injections, and include without limitation intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The pharmaceutical compositions may be formulated according to conventional pharmaceutical practice (see, e.g., Remington: The Science and Practice of Pharmacy (20th ed.), Ed. AR Gennaro, Lippincott Williams & Wilkins, 2000 and Encyclopedia of Pharmaceutical Technology, eds J. Swarbrick and J C Boylan, 1988-1999, Marcel Dekker, New York).

Formulations suitable for oral administration can be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations can be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet can be prepared by compressing or molding a powder or granule containing the active agent, optionally with one or more accessory ingredients. Compressed tablets can be prepared by compressing, in a suitable machine, the compound in a free-flowing form, such as a powder or granules optionally mixed with a binder, lubricant, inert diluent, and/or surface active/dispersing agent(s). Molded tablets can be made by molding, in a suitable machine, the powdered compound moistened with an inert liquid binder.

Formulations suitable for buccal (sub-lingual) administration include lozenges having the active agent in a flavored base, usually sucrose and acacia or tragacanth; and pastilles containing the active agent in an inert base such as gelatin and glycerin or sucrose and acacia.

Formulations for parenteral administration are conveniently sterile aqueous preparations of the active agent, which preparations are preferably isotonic with the blood of the intended recipient. These preparations can be administered by means of subcutaneous, intravenous, intramuscular, or intradermal injection. Such preparations can conveniently be prepared by admixing the compound with water or a glycine buffer and rendering the resulting solution sterile and isotonic with the blood.

Formulations suitable for topical application (e.g., in the oral passage, nasopharynx, or oropharynx) take the form of an ointment, cream, lotion, paste, gel, spray, aerosol, or oil. Carriers which can be used include vaseline, lanoline, polyethylene glycols, alcohols, transdermal enhancers, and combinations of two or more thereof.

In some embodiment, the disclosure provides a method of treatment wherein the compositions provided herein are administered in combination with one or more additional therapeutic agent(s). The combination of the provided compositions and therapeutic agent(s) may be administered or co-administered (e.g., consecutively, simultaneously, at different times) in any conventional dosage form. Co-administration in the present context refers to the administration of more than one therapeutic agent to a subject in the course of a coordinated treatment to achieve an improved clinical outcome. Such co-administration may also be coextensive, that is, occurring during overlapping periods of time. For example, a first Therapeutic agent may be administered to a patient before, concomitantly, before and after, or after a second active agent is administered. In some embodiments the Therapeutic agents are combined/formulated in a single composition and thus administered to the subject at the same time.

### Methods of Treatment and Use

### Cardiovascular Disease

The disclosure generally provides methods and compositions for treating a cardiovascular disease.

In one embodiment, the disclosure provides a method of treating an cardiovascular disease in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the cardiovascular disease treated according to the provided methods and compositions is selected from: coronary artery disease (CAD) (such as angina and myocardial infarction, coronary heart disease (e.g., ischemic heart disease), acute coronary syndrome, angina, heart failure, aortic aneurysm, aortic dissection, iliac or femoral aneurysm, pulmonary embolism, primary hypertension, atrial fibrillation, stroke, transient ischemic attack, systolic dysfunction, diastolic dysfunction, carditis (e.g., endocarditis, myocarditis, acute myocarditis, acute pericarditis and complicated pericarditis), atrial tachycardia, ventricular fibrillation, cardiac allograft rejection arteriopathy, vasculitis, thrombosis, atherosclerosis, atherosclerotic plaque, vulnerable plaque, acute coronary syndrome, acute ischemic attack, sudden cardiac death, cerebrovascular disease, peripheral vascular disease, peripheral artery disease (PAD), and cerebrovascular disease.

In some embodiments, the CVD treated according to the provided methods and compositions is selected from: acute coronary syndrome, coronary artery disease, myocardial infarction, coronary heart disease, carditis, an ischemic cardiovascular disease, heart failure, stroke, peripheral vascular disease and ischemia/reperfusion injury.

In some embodiments, the CVD treated according to the provided methods and compositions an ischemic CVD. In some embodiments, the treated CVD is myocardial ischemia. In some embodiments, the CVD treated according to the provided methods is myocardial infarction. In some embodiments, the CVD treated according to the provided methods is stroke.

In some embodiments, the CVD treated according to the provided methods is ischemic cardiovascular disease. Ischemic cardiovascular disease is a cardiovascular disease, which arises from the vessel occlusion due to any cause such as thrombus formation, and specifically, myocardial infarction or angina pectoris.

In some embodiments, the provided methods and compositions treat ischemic heart disease. Ischemic heart disease is characterized by a reduced blood supply of heart muscle, usually due to atherosclerosis. Signs and symptoms of ischemic heart disease include angina pectoris (chest pain on exertion, in cold weather or emotional situations), acute chest pain (i.e., heart attack) such as acute coronary syndrome, unstable angina or myocardial infarction, heart failure with associated difficulty in breathing or swelling of the extremities, and heartburn. Risk factors for ischemic heart disease include age, smoking, hypercholesterolemia, diabetes, and hypertension.

In some embodiments, the CVD treated according to the provided methods and compositions is myocardial ischemia (MI). MI is an aspect of heart dysfunction that occurs when the heart muscle (the myocardium) does not receive adequate blood supply and is thus deprived of necessary levels of oxygen and nutrients. Myocardial ischemia may result in a variety of heart diseases including, for example, angina, heart attack and/or heart failure.

In some embodiments, the CVD treated according to the provided methods and compositions is cerebrovascular disease. Cerebrovascular disease refers to brain dysfunctions related to disease of the blood vessels supplying the brain.

In some embodiments, the provided methods and compositions treat heart disease (HD). HD refers to acute and/or chronic cardiac dysfunctions. Heart disease is often associated with a decrease in cardiac contractile function and may be associated with an observable decrease in blood flow to the myocardium (e.g., as a result of coronary artery disease). Manifestations of heart disease include myocardial ischemia, which may result in angina, heart attack and/or congestive heart failure. Congestive heart failure is defined as abnormal heart function resulting in inadequate cardiac output to meet metabolic needs.

In some embodiments, the CVD treated according to the provided methods and compositions is peripheral vascular disease (PVD). PVD refers to acute or chronic dysfunction of the peripheral (i.e., non-cardiac) vasculature and/or the tissues supplied thereby. As with heart disease, peripheral vascular disease typically results from an inadequate blood flow to the tissues supplied by the vasculature, which lack of blood may result, for example, in ischemia or, in severe cases, in tissue cell death. Aspects of peripheral vascular disease include, without limitation, peripheral arterial occlusive disease (PAOD) and peripheral muscle ischemia. Frequently, symptoms of peripheral vascular disease are manifested in the extremities of the patient, especially the legs.

In some embodiments, the CVD treated according to the provided methods and compositions is ischemia/reperfusion injury.

In some embodiments, the subject treated according to the provided methods is at risk of having an cardiovascular disease. In some embodiments, a method provided herein, is performed as a prophylactic treatment for a cardiovascular disease.

In some embodiments, the provided methods and compositions prevent a cardiovascular disease in a subject at risk for developing the cardiovascular disease, e.g., a subject having one or more risk factors associated with development of the cardiovascular disease. In some embodiments, the subject has one or more risk factors selected from hyperglycemia, hypertension, hyperlipidemia, high total cholesterol, low HDL cholesterol, high average systolic blood pressure and high hemoglobin A1c a family history of CVD, smoking, renal disease, obesity, diabetes (e.g., advanced diabetes).

In some embodiments, the subject treated according to the provided methods has a CVD. In some embodiments, the subject has been diagnosed as having a CVD (e.g., heart failure or a coronary artery disease (CAD) such as angina or myocardial infarction). The development of cardiovascular disease such as heart failure and CAD can routinely be detected and assessed using standard clinical techniques known in the art, such as echocardiography and biomarkers.

In some embodiments, the disclosure provides methods and compositions that prevent, inhibit or delay the onset of an cardiovascular disease by administering compositions provided herein to a subject before the onset of the cardiovascular disease, e.g., before the onset of one or more symptoms thereof.

In some embodiments, the provided methods prevent, reduce or delay the cardiovascular disease. The methods may be administered to patients at risk for developing the cardiovascular disease. In such subjects, prevention of an cardiovascular disease may be monitored by for example, angiography, electrocardiography (ECG), or by lack of typical hallmarks of the cardiovascular disease. For example, subjects to whom an effective amount of a HIF1-alpha inhibitor and PFKFB3 inhibitor is administered prophylactically may not experience or may experience a reduced incidence of one or more of angina, chest pain, nausea, indigestion, shortness of breath, sudden heavy sweating, lightheadedness, dizziness or fainting; unusual fatigue; and a feeling of restlessness or apprehension.

In some embodiments, treating a cardiovascular disease according to a method provided herein comprises delaying the onset of one or more symptoms of a cardiovascular disease such as, angina, cardiovascular ischemia, myocardial infarction, stroke, heart failure, and/or reperfusion injury.

In some embodiments, the HIF1-α Pathway Inhibitor and the PFKFB3 inhibitor are administered after the onset of one or more symptoms of a cardiovascular disease. In some embodiments, the inhibitors are administered after the subject has experienced, or has been diagnosed as having experienced, a cardiovascular event such as, myocardial infarction or ischemia/reperfusion injury. In some embodiments, the inhibitors are administered after the subject has experienced, or has been diagnosed as having experienced a cardiovascular event (e.g., angina, myocardial infarction or ischemia/reperfusion injury). In some embodiments, the inhibitors are administered after the subject has experienced myocardial infarction or a stroke. In some embodiments, the inhibitors are administered after ischemia/reperfusion injury. In some embodiments, the provided methods and compositions are used to treat different stages of a cardiovascular disease.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is antisense oligonucleotide EZN-2968 or nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In a particular embodiment, the PFKFB3 inhibitor administered according to the provided methods is AZ67, or a salt thereof.

In some embodiments, the PFKFB3 inhibitor administered according to the provided methods is at least one PFKFB3 inhibitor having the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In other embodiments, the PFKFB3 inhibitor administered according to the provided methods is the PFKFB3 inhibitor having the structure of formula AZ44-AZ70 or AZ71, depicted in FIG. 1E, or a salt thereof.

In some embodiments, a method provided herein for treating cardiovascular disease is performed by co-administering the HIF1-α Pathway Inhibitor and the PFKFB3 inhibitor to the subject.

In some embodiments, the administration of the HIF1-α Pathway Inhibitor and/or the PFKFB3 inhibitor is administered orally, transmucosal administration, syrup, topical administration, parenteral administration, injection, subdermal administration, rectal administration, buccal administration or transdermal administration. The phrases “parenteral administration” and “administered parenterally” as used herein refer to modes of administration other than enteral and topical administration, such as injections, and include without limitation intravenous, intramuscular, intrapleural, intravascular, intrapericardial, intraarterial, intrathecal, intracapsular, intraorbital, intracardiac, intradennal, intraperitoneal, transtracheal, subcutaneous, subcuticular, intra-articular, subcapsular, subarachnoid, intraspinal and intrastemal injection and infusion.

In some embodiments, treating a cardiovascular disease according to a method provided herein comprises reducing one or more symptoms of the cardiovascular disease in the subject compared to the subject prior to treatment with the HIF1-α Pathway Inhibitor and the PFKFB3 inhibitor. In some embodiments, the one or more reduced symptoms of the cardiovascular disease is selected from: reduction in apoptosis/destruction (i.e., loss of) of cardiovascular cells and/or tissue (e.g., endothelial cells, cardiomyocytes, and heart); increase in survival and/or function of cardiovascular cells and/or tissue (e.g., endothelial cells, cardiomyocytes, and heart); reduction in long-term damage to cardiovascular cells/tissue and/or to surrounding cells/tissue; decrease of the inflammation in cardiovascular cells/tissues; reduction in the oxidative stress in cardiovascular cells/tissues; and increased survival/survival time. In some embodiments, the one or more reduced symptoms of the cardiovascular disease is selected from reduction in: heart and/or respiratory rate, rales, edema, jugular venous distension, the expression one or more biomarkers, and/or enlargement of the heart. In some embodiments, the one or more symptoms of cardiovascular disease are reduced by at least 10%, at least 20%, at least 30%, at least 40%, or at least 50% compared the subject prior to treatment with the HIF1-α Pathway Inhibitor and the PFKFB3 inhibitor.

Treatment and/or prevention of a cardiovascular disease can be measured by a variety of means. In some embodiments, treatment or prevention comprises treating one or more of infarct, apoptosis, inflammation, and compromised vascular endothelial cell and/or cardiomyocyte function in a subject.

In some embodiments, the provided methods result in decreased apoptosis and/or death of vascular endothelial cells and/or cardiomyocytes in the subject. Cell death can be monitored according to known methods. Illustrative methods for detecting cell death include but are not limited to, nuclear staining techniques such as propidium iodide, Hoechst-33342, 4′,6-diamidino-2-phenylindole (DAPI), and Acridine orange-Ethidium bromide staining. Nonnuclear staining techniques include but are not limited to, Annexin-V staining. In some embodiments, the provided methods result in increased injury recovery and/or function of vascular endothelial cells and/or cardiomyocytes in the subject. Techniques for assessing the recovery and function of vascular endothelial cells and cardiomyocytes are known in the art.

In some embodiments, the provided methods reduce levels of inflammatory cytokines such as, TNFα, IL-1β, IL-6, or MCP1 in a biological sample from the subject. Cytokine levels in the subject can routinely be monitored via enzyme-linked immunosorbant assay (ELISA), Luminex, Cytokine Bead Array, Proteo Plex, FAST Quant, other techniques known in the art.

In some embodiments, the disclosure provides further administering an additional therapeutic agent to the subject.

### Acute Coronary Syndrome (ACS)

In some embodiments, the disclosure provides methods and compositions for treating acute coronary syndrome” (“ACS”). ACS is a group of coronary artery diseases resulting from ischemic injury to the heart, which is generally dependent on atherosclerosis and hypertension and is the leading cause of death in the United States. ACS patients form heterogeneous groups that differ in pathophysiology, clinical status, and risk of adverse events. ACS can manifest as stable angina, unstable angina, or myocardial infarction. ACS subjects may have unstable angina, non-ST-elevation (NST) non-Q wave myocardial infarction (MI), ST-elevation non-Q wave MI, or transmural (Q wave).

Stable angina is characterized by constricted chest pain caused by intense activity and stress, and is relieved by rest and sublingual nitroglycerin. Unstable angina is believed to represent a clinical condition between stable angina and myocardial infarction, and is usually associated with atherosclerotic plaque rupture and thrombus formation. Unstable angina is characterized by stenotic chest pain at rest that is alleviated by sublingual nitroglycerin. Myocardial infarction is characterized by constricted chest pain lasting more than 30 minutes, which can be accompanied by diagnostic electrocardiogram (ECG) Q wave.

In some embodiments, the disclosure provides methods and compositions for treating ACS in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the subject is at risk of having ACS. In some embodiments, a method provided herein (e.g., any of (a)-(c) above), is performed as a prophylactic treatment for ACS.

In some embodiments, the provided methods and compositions prevent ACS in a subject at risk for developing ACS, e.g., a subject having one or more risk factors associated with development of ACS. In some embodiments, the subject has one or more risk factors selected from: over 60 years of age, smoking, obesity, poor diet, hyperglycemia, hypertension, hyperlipidemia, renal disease, and diabetes. In some embodiments, the subject has one or more risk factors selected from: high average systolic blood pressure and high hemoglobin A1c.

In some embodiments, the disclosure provides methods and compositions that prevent, inhibit or delay the onset of ACS by administration of the provided compositions to a subject before the onset of ACS, e.g., before the onset of one or more symptoms of ACS.

In some embodiments, the HIF1-α Pathway Inhibitor and the PFKFB3 inhibitor are administered before the onset of one or more symptoms of ACS. In some embodiments, the provided methods prevent ACS. In some embodiments, the provided methods delay the onset of ACS.

In some embodiments, the provided methods are administered to a subject at risk for developing ACS. In such subjects, prevention of ACS may be monitored by lack of typical hallmarks of ACS. For example, subjects to whom an effective amount of a HIF1-alpha inhibitor and PFKFB3 inhibitor is administered prophylactically may not experience or may experience a reduced incidence of one or more of the following symptoms: angina, chest pain spreading from the chest to the shoulders, arms, upper abdomen, back, neck or jaw; nausea or vomiting, indigestion, shortness of breath (dyspnea); sudden, heavy sweating (diaphoresis); lightheadedness, dizziness or fainting; unusual fatigue; and a feeling of restlessness or apprehension. In some embodiments, the subject's serum does not have elevated levels of 1 or more ACS biomarkers (e.g., creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin).

In some embodiments, the subject has been diagnosed as having ACS. Current methods for diagnosing and monitoring ACS generally include clinical symptoms, electrocardiography (ECG), and measurement of peripheral circulation heart biomarkers. Angiography is also used for severe chest pain usually associated with unstable angina and acute myocardial infarction (AMI). Patients with ACS often have constricted chest pain that often spreads inside the neck, chin, shoulders, or left or both arms, and may be accompanied by symptoms of dyspnea, sweating, palpitation, head wandering, and nausea. Myocardial ischemia can cause changes in the diagnostic ECG, such as changes in the Q wave and ST segment. Elevated plasma concentrations of cardiac enzymes of the subject reflect the degree of cardiac tissue necrosis associated with severe unstable angina and myocardial infarction.

In some embodiments, the HIF1-α Pathway Inhibitor and the PFKFB3 inhibitor are administered after the onset of one or more symptoms of ACS. In some embodiments, the subject exhibits at least one of the following: angina, chest pain spreading from the chest to the shoulders, arms, upper abdomen, back, neck or jaw; nausea or vomiting, indigestion, shortness of breath (dyspnea); sudden, heavy sweating (diaphoresis); lightheadedness, dizziness or fainting; unusual fatigue; and a feeling of restlessness or apprehension. In some embodiments, the provided methods and compositions may reduce the incidence, severity, or level of one or more of the above symptoms.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is antisense oligonucleotide EZN-2968 or nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In a particular embodiment, the PFKFB3 inhibitor administered according to the provided methods is AZ67, or a salt thereof.

In some embodiments, the PFKFB3 inhibitor administered according to the provided methods is at least one PFKFB3 inhibitor having the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In other embodiments, the PFKFB3 inhibitor administered according to the provided methods is the PFKFB3 inhibitor having the structure of formula AZ44-AZ70 or AZ71, depicted in FIG. 1E, or a salt thereof.

In some embodiments, a method provided herein for treating ACS is performed by co-administering the HIF1-α Pathway Inhibitor and the PFKFB3 inhibitor to the subject.

In some embodiments, the administration of the HIF1-α Pathway Inhibitor and/or the PFKFB3 inhibitor is administered orally. In some embodiments, the administration of the HIF1-α Pathway Inhibitor and/or the PFKFB3 inhibitor is administered, via transmucosal administration, syrup, topical t administration, parenteral administration, injection, subdermal administration, rectal administration, buccal administration or transdermal administration.

In some embodiments, treating ACS according to a method provided herein comprises reducing one or more symptoms of ACS in the subject compared to a control subject or compared to the subject prior to treatment with the HIF1-α Pathway Inhibitor and the PFKFB3 inhibitor. In some embodiments, the one or more reduced symptoms of ACS is selected from: angina, chest pain spreading from the chest to the shoulders, arms, upper abdomen, back, neck or jaw; nausea or vomiting, indigestion, shortness of breath (dyspnea); sudden, heavy sweating (diaphoresis); lightheadedness, dizziness or fainting; unusual fatigue; and a feeling of restlessness or apprehension. In some embodiments, the provided methods result in a normalized ECG (e.g., reversion of changes in the Q wave and ST segment to normal) or reduced levels of plasma concentrations of cardiac enzymes or other biomarkers (e.g., creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin). In some embodiments, the one or more symptoms of ACS are reduced by at least 10%, at least 20%, at least 30%, at least 40%, or at least 50% compared to the control subject or compared to the subject prior to treatment with the HIF1-α Pathway Inhibitor and the PFKFB3 inhibitor.

In some embodiments, 1, 2, 3, 4, 5, or more ACS biomarkers (e.g., creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin) in a biological sample of the subject is reduced by at least 10%, at least 20%, at least 30%, at least 40%, or at least 50% compared to a control subject or compared to the subject prior to treatment with the HIF1-α Pathway Inhibitor and the PFKFB3 inhibitor.

In additional embodiments, the provided methods include further administering an additional therapeutic agent to the subject.

### Myocardial Infarction (MI)

“Infarct” or “infarction” relates to a localized area of ischemic necrosis produced by anoxia following occlusion of the arterial supply or venous drainage of a tissue or organ. A myocardial infarction (MI), involves an ischemic necrosis of part of the myocardium due to the obstruction of one or several coronary arteries or their branches. Myocardial infarction is characterized by the loss of functional cardiomyocytes, the myocardial tissue being irreversibly damaged. The myocardium, or heart muscle, suffers an infarction when advanced coronary disease exists.

In some embodiments, the disclosure provides methods and compositions for treating MI in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the subject is at risk of having MI. In some embodiments, a method provided herein (e.g., any of (a)-(c) above), is performed as a prophylactic treatment for MI.

In some embodiments, the provided methods and compositions prevent MI in a subject at risk for developing MI, e.g., a subject having one or more risk factors associated with development of MI. In some embodiments, the subject has one or more risk factors selected from: over 60 years of age, previous cardiovascular disease, family history of premature myocardial infarction, tobacco smoking, diabetes, high blood pressure, lack of physical activity, obesity, chronic kidney disease, advanced coronary disease body mass index, physical activity, non-fasting total cholesterol, HDL cholesterol, LDL cholesterol, and triglycerides, and excessive alcohol consumption

In some embodiments, the disclosure provides methods and compositions that prevent, inhibit or delay the onset of MI by administration of the provided compositions to a subject before the onset of MI, e.g., before the onset of one or more symptoms of mi.

In some embodiments, the HIF1-α Pathway Inhibitor and the PFKFB3 inhibitor are administered before the onset of one or more symptoms of MI. In some embodiments, the provided methods prevent MI. In some embodiments, the provided methods delay the onset of MI. In some embodiments, the provided methods are administered to a subject at risk for developing MI. In such subjects, prevention of MI may be monitored by lack of typical hallmarks of MI. For example, subjects to whom an effective amount of a HIF1-alpha inhibitor and PFKFB3 inhibitor is administered prophylactically may not experience or may experience a reduced incidence of one or more of the following symptoms: unstable angina; nausea or vomiting, indigestion, dyspnea; diaphoresis; lightheadedness, dizziness or fainting; unusual fatigue; and a feeling of restlessness or apprehension. In some embodiments, the subject's serum does not have elevated levels of 1 or more biomarkers selected from creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin, or elevated levels of cardiac enzymes.

In some embodiments, the subject has been diagnosed as having MI. Current methods for diagnosing and monitoring MI generally include clinical symptoms, electrocardiography (ECG), and measurement of peripheral circulation heart biomarkers. Angiography is also used for severe chest pain usually associated with unstable angina and myocardial infarction. Patients with MI often have constricted chest pain that often spreads inside the neck, chin, shoulders, or left or both arms, and may be accompanied by symptoms of dyspnea, sweating, palpitation, head wandering, and nausea. Myocardial ischemia can cause changes in the diagnostic ECG, such as changes in the Q wave and ST segment. Elevated plasma concentrations of cardiac enzymes reflect the degree of cardiac tissue necrosis associated with severe unstable angina and myocardial infarction.

In some embodiments, the HIF1-α Pathway Inhibitor and the PFKFB3 inhibitor are administered after the onset of one or more symptoms of MI. In some embodiments, the subject exhibits at least one of the following: angina, chest pain spreading from the chest to the shoulders, arms, upper abdomen, back, neck or jaw; nausea or vomiting; indigestion; dyspnea; diaphoresis; lightheadedness, dizziness or fainting; unusual fatigue; and a feeling of restlessness or apprehension. In some embodiments, the provided methods and compositions may reduce the incidence, severity, or level of one or more of the above symptoms.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is antisense oligonucleotide EZN-2968 or nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In a particular embodiment, the PFKFB3 inhibitor administered according to the provided methods is AZ67, or a salt thereof.

In some embodiments, the PFKFB3 inhibitor administered according to the provided methods is at least one PFKFB3 inhibitor having the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In other embodiments, the PFKFB3 inhibitor administered according to the provided methods is the PFKFB3 inhibitor having the structure of formula AZ44-AZ70 or AZ71, depicted in FIG. 1E, or a salt thereof.

In some embodiments, a method provided herein for treating MI is performed by co-administering the HIF1-α Pathway Inhibitor and the PFKFB3 inhibitor to the subject.

In some embodiments, the administration of the HIF1-α Pathway Inhibitor and/or the PFKFB3 inhibitor is administered orally. In some embodiments, the administration of the HIF1-α Pathway Inhibitor and/or the PFKFB3 inhibitor is administered, via transmucosal administration, syrup, topical administration, parenteral administration, injection, subdermal administration, rectal administration, buccal administration or transdermal administration.

In some embodiments, treating a MI disease according to a method provided herein comprises reducing one or more symptoms of the cardiovascular disease in the subject compared to the subject prior to treatment with the HIF1-α Pathway Inhibitor and the PFKFB3 inhibitor. In some embodiments, the provided methods result in a reduced apoptosis/destruction (i.e., loss of) of or injury to cardiomyocyte cells and/or tissue (e.g., heart); increased survival and/or function of cardiomyocytes and the heart; reduced long-term damage to cardiomyocytes and surrounding cells/tissue; decrease of the inflammation in cardiovascular cells/tissues; reduction in the oxidative stress in cardiovascular cells/tissues; and increased survival/survival time. In some embodiments, the provided methods result in a reduced are of cardiac tissue and/or infarct size. In some embodiments, the one or more symptoms of cardiovascular disease are reduced by at least 10%, at least 20%, at least 30%, at least 40%, or at least 50% compared the subject prior to treatment with the HIF1-α Pathway Inhibitor and the PFKFB3 inhibitor.

In some embodiments, treating MI according to a method provided herein comprises reducing one or more symptoms of MI in the subject compared to a control subject or compared to the subject prior to treatment with the HIF1-α Pathway Inhibitor and the PFKFB3 inhibitor. In some embodiments, the one or more reduced symptoms of MI is selected from: angina, chest pain spreading from the chest to the shoulders, arms, upper abdomen, back, neck or jaw; nausea or vomiting, indigestion, shortness of breath (dyspnea); sudden, heavy sweating (diaphoresis); lightheadedness, dizziness or fainting; unusual fatigue; and a feeling of restlessness or apprehension.

In some embodiments, the provided methods result in a normalized ECG (e.g., reversion of changes in the Q wave and ST segment to normal) or reduced levels of plasma concentrations of cardiac enzymes or other biomarkers (e.g., creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin). In some embodiments, the one or more symptoms of MI are reduced by at least 10%, at least 20%, at least 30%, at least 40%, or at least 50% compared to the control subject or compared to the subject prior to treatment with the HIF1-α Pathway Inhibitor and the PFKFB3 inhibitor. In some embodiments, 1, 2, 3, 4, 5, or more ACS biomarkers (e.g., creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin) in a biological sample of the subject is reduced by at least 10%, at least 20%, at least 30%, at least 40%, or at least 50% compared to the control subject or compared to the subject prior to treatment with the HIF1-α Pathway Inhibitor and the PFKFB3 inhibitor. ECG testing can be used to determine if the MI is an ST elevation MI (STEMI) which usually requires more aggressive treatment. Methods to determine infarct size are known in the art and include without limitation, measurement of serum markers such as creatine kinase (CK)-MB levels in a serum sample, tissue staining with triphenyl tetrazolium chloride, technetium (Tc)-99m sestamibi single-photon emission computed tomography (SPECT) myocardial perfusion imaging, and magnetic resonance.

In additional embodiments, the provided methods include further administering an additional therapeutic agent to the subject.

### Heart Failure

Heart failure (HF), often called congestive heart failure, is a clinical syndrome characterized by the inability of the heart to supply sufficient blood flow to meet the metabolic demands of the body. Common causes of HF include myocardial infarction and other forms of ischemic heart disease, hypertension, valvular heart disease, and cardiomyopathy.

In some embodiments, the disclosure provides methods and compositions for treating HF in a subject comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the subject is at risk of having HF. In some embodiments, a method provided herein (e.g., any of (a)-(c) above), is performed as a prophylactic treatment for HF.

In some embodiments, the provided methods and compositions prevent HF in a subject at risk for developing HF, e.g., a subject having one or more risk factors associated with development of HF. In some embodiments, the subject has one or more risk factors selected from: over 60 years of age, smoking, obesity, metabolic syndrome, hypertension (e.g., arterial hypertension), coronary artery disease, diabetes mellitus, family history of cardiomyopathy, valvular heart disease, and use of cardiotoxins. In some embodiments, the subject has one or more risk factors selected from high average systolic blood pressure and high hemoglobin A1c.

In some embodiments, the disclosure provides methods and compositions that prevent, inhibit or delay the onset of HF by administration of the provided compositions to a subject before the onset of HF, e.g., before the onset of one or more symptoms of HF.

In some embodiments, the HIF1-α Pathway Inhibitor and the PFKFB3 inhibitor are administered before the onset of one or more symptoms of HF. In some embodiments, the provided methods prevent HF. In some embodiments, the provided methods delay the onset of HF. In some embodiments, the provided methods are administered to a subject at risk for developing HF. In such subjects, prevention of HF may be monitored by lack of typical hallmarks of HF. For example, subjects to whom an effective amount of a HIF1-alpha inhibitor and PFKFB3 inhibitor is administered prophylactically may not experience or may experience a reduced incidence of one or more of the following symptoms: shortness of breath, fatigue, weakness, leg swelling, exercise intolerance, elevations in heart and respiratory rates, rales (an indication of fluid in the lungs), edema, jugular venous distension, and an enlarged heart.

In some embodiments, the subject has been diagnosed as having HF. Current methods for diagnosing and monitoring HF generally include clinical symptoms, electrocardiography (ECG), and measurement of peripheral circulation heart biomarkers.

In some embodiments, the HIF1-α Pathway Inhibitor and the PFKFB3 inhibitor are administered after the onset of one or more symptoms of HF. In some embodiments, the subject exhibits at least one of the following: shortness of breath, fatigue, weakness, leg swelling, exercise intolerance, elevations in heart and respiratory rates, rales (an indication of fluid in the lungs), edema, jugular venous distension, and an enlarged heart. In some embodiments, the provided methods and compositions may reduce the incidence, severity, or level of one or more of the above symptoms.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment. Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is antisense oligonucleotide EZN-2968 or nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In a particular embodiment, the PFKFB3 inhibitor administered according to the provided methods is AZ67, or a salt thereof.

In some embodiments, the PFKFB3 inhibitor administered according to the provided methods is at least one PFKFB3 inhibitor having the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In other embodiments, the PFKFB3 inhibitor administered according to the provided methods is the PFKFB3 inhibitor having the structure of formula AZ44-AZ70 or AZ71, depicted in FIG. 1E, or a salt thereof.

In some embodiments, a method provided herein for treating HF is performed by co-administering the HIF1-α Pathway Inhibitor and the PFKFB3 inhibitor to the subject.

In some embodiments, the administration of the HIF1-α Pathway Inhibitor and/or the PFKFB3 inhibitor is administered orally. In some embodiments, the administration of the HIF1-α Pathway Inhibitor and/or the PFKFB3 inhibitor is administered, via transmucosal administration, syrup, topical administration, parenteral administration, injection, subdermal administration, rectal administration, buccal administration or transdermal administration.

In some embodiments, treating HF according to a method provided herein comprises reducing one or more symptoms of HF in the subject compared to a control subject or compared to the subject prior to treatment with the HIF1-α Pathway Inhibitor and the PFKFB3 inhibitor. In some embodiments, the one or more reduced symptoms of HF is selected from: shortness of breath, fatigue, weakness, leg swelling, exercise intolerance, elevations in heart and respiratory rates, rales (an indication of fluid in the lungs), edema, jugular venous distension, and an enlarged heart. In some embodiments, the one or more symptoms of HF are reduced by at least 10%, at least 20%, at least 30%, at least 40%, or at least 50% compared to the control subject or compared to the subject prior to treatment with the HIF1-α Pathway Inhibitor and the PFKFB3 inhibitor.

In some embodiments, 1, 2, 3, 4, 5, or more HF biomarkers (e.g., plasma hsCRP, IL-1beta and IL-6, and/or B-type Natriuretic Peptide (BNP)) in a biological sample of the subject is reduced by at least 10%, at least 20%, at least 30%, at least 40%, or at least 50% compared to the control subject or compared to the subject prior to treatment with the HIF1-α Pathway Inhibitor and the PFKFB3 inhibitor.

In some embodiments, the administration of the HIF1-α Pathway Inhibitor and/or the PFKFB3 inhibitor results in the subject having one or more of improved CPX scores, improved ECG recordings, and/or improved bioimpedance analysis, and/or has a reduced risk of being re-hospitalized for an indication associated with heart failure

In additional embodiments, the provided methods include further administering an additional therapeutic agent to the subject.

### Stroke

The term “stroke” refers to the sudden death of brain cells due to a lack of oxygen when the blood flow to the brain is impaired by blockage or rupture of an artery to the brain. Strokes can be classified into two major categories: ischemic and hemorrhagic. Ischemic strokes are those that are caused by interruption of the blood supply, while hemorrhagic strokes are the ones which result from rupture of a blood vessel or an abnormal vascular structure In an ischemic stroke, blood supply to part of the brain is decreased as a result of thrombosis (obstruction of a blood vessel by a blood clot forming locally), embolism (obstruction due to an embolus from elsewhere in the body, systemic hypoperfusion (general decrease in blood supply, e.g., in shock), or venous thrombosis.

In some embodiments, the disclosure provides methods and compositions for treating stroke in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, the subject is at risk of having stroke. In some embodiments, a method provided herein (e.g., any of (a)-(c) above), is performed as a prophylactic treatment for stroke.

In some embodiments, the provided methods and compositions prevent stroke in a subject at risk for developing stroke, e.g., a subject having one or more risk factors associated with development of stroke. In some embodiments, the subject has one or more risk factors selected from: over 60 years of age, high blood pressure, previous stroke or transient ischemic attack, diabetes, obesity, high cholesterol, smoking and atrial fibrillation.

In some embodiments, the disclosure provides methods and compositions that prevent, inhibit or delay the onset of stroke by administration of the provided compositions to a subject before the onset of stroke, e.g., before the onset of one or more symptoms of stroke.

In some embodiments, the HIF1-α Pathway Inhibitor and the PFKFB3 inhibitor are administered before the onset of one or more symptoms of stroke. In some embodiments, the provided methods prevent stroke. In some embodiments, the provided methods delay the onset of stroke. In some embodiments, the provided methods are administered to a subject at risk for developing stroke. In such subjects, prevention of stroke may be monitored by lack of typical hallmarks of stroke.

In some embodiments, the subject has been diagnosed as having stroke. Current methods for diagnosing and monitoring stroke generally include clinical symptoms, for stroke include noncontrast computed tomography (CT) scan, magnetic resonance imaging (MRI), and angiography electrocardiography (ECG), and measurement of peripheral circulation heart biomarkers.

Patients suffering a stroke often have sudden numbness or weakness in the face, arm, or leg, especially on one side of the body; sudden confusion, trouble speaking, or difficulty understanding speech; Sudden trouble seeing in one or both eyes; and/or sudden trouble walking, dizziness, loss of balance, or lack of coordination.

In some embodiments, the HIF1-α Pathway Inhibitor and the PFKFB3 inhibitor are administered after the onset of one or more symptoms of stroke. In some embodiments, the subject has exhibited at least one of the following: sudden numbness or weakness in the face, arm, or leg, especially on one side of the body; sudden confusion, trouble speaking, or difficulty understanding speech; Sudden trouble seeing in one or both eyes; and/or sudden trouble walking, dizziness, loss of balance, or lack of coordination.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is antisense oligonucleotide EZN-2968 or nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In a particular embodiment, the PFKFB3 inhibitor administered according to the provided methods is AZ67, or a salt thereof.

In some embodiments, the PFKFB3 inhibitor administered according to the provided methods is at least one PFKFB3 inhibitor having the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In other embodiments, the PFKFB3 inhibitor administered according to the provided methods is the PFKFB3 inhibitor having the structure of formula AZ44-AZ70 or AZ71, depicted in FIG. 1E, or a salt thereof.

In some embodiments, a method provided herein for treating stroke is performed by co-administering the HIF1-α Pathway Inhibitor and the PFKFB3 inhibitor to the subject.

In some embodiments, the administration of the HIF1-α Pathway Inhibitor and/or the PFKFB3 inhibitor is administered orally. In some embodiments, the administration of the HIF1-α Pathway Inhibitor and/or the PFKFB3 inhibitor is administered, via transmucosal administration, syrup, topical administration, parenteral administration, injection, subdermal administration, rectal administration, buccal administration or transdermal administration.

In some embodiments, treating a stroke disease according to a method provided herein comprises reducing one or more symptoms of the cardiovascular disease in the subject compared to the subject prior to treatment with the HIF1-α Pathway Inhibitor and the PFKFB3 inhibitor. In some embodiments, the provided methods result in a reduced apoptosis/destruction (i.e., loss of) of or injury to endothelial cells, neural cells, and/or tissue (e.g., neural tissue); increased survival and/or function of vascular endothelial cells and/or neural cells; reduced in long-term damage to vascular endothelial cells, neural cells, and surrounding cells/tissue; decrease of the inflammation in vascular endothelial and/or neural cells/tissues; reduction in the oxidative stress in vascular endothelial and/or neural cells; and increased survival/survival time.

In some embodiments, the provided methods result in a reduced lesion volume, reduced brain inflammatory levels, increased probability of recovery on the mRS score, and/or reduced cytotoxic edema. In some embodiments, the provided methods result in a reduced lesion volume of at least 10%, at least 20%, at least 30%, at least 40%, or at least 50% compared the subject prior to treatment with the HIF1-α Pathway Inhibitor and the PFKFB3 inhibitor.

In some embodiments, treating stroke according to a method provided herein comprises reducing one or more symptoms of stroke in the subject compared to a control subject or compared to the subject prior to treatment with the HIF1-α Pathway Inhibitor and the PFKFB3 inhibitor. In some embodiments, the one or more reduced symptoms of stroke is selected from: numbness or weakness in the face, arm, or leg, especially on one side of the body; confusion, trouble speaking, or difficulty understanding speech; trouble seeing in one or both eyes; and/or trouble walking, dizziness, loss of balance, or lack of coordination.

In some embodiments, 1, 2, 3, 4, 5, or more serum biomarkers (e.g., E-selectin, ICAM-1, VCAM, and MCP-1) of the subject is reduced by at least 10%, at least 20%, at least 30%, at least 40%, or at least 50% compared to the control subject or compared to the subject prior to treatment with the HIF1-α Pathway Inhibitor and the PFKFB3 inhibitor.

In additional embodiments, the provided methods include further administering an additional therapeutic agent to the subject. In one embodiment, the additional administered therapeutic agent is tissue plasminogen activator (TPA), an anticoagulant (e.g., heparin).

### Ischemia/Reperfusion Injury

Ischemia, the lack of oxygen to an organ, rapidly sets into motion a complex series of events that affect the structure and function of virtually every organelle and subcellular system of the affected cells. Ischemia/reperfusion injury leads to production of excessive amounts of reactive oxygen species (ROS) and reactive nitrogen species (RNS) causing oxidative stress which results in alterations in mitochondrial oxidative phosphorylation, depletion of ATP, an increase in intracellular calcium and activation of protein kinases, phosphatases, proteases, lipases and nucleases leading to loss of cellular function/integrity.

In some embodiments, the disclosure provides methods and compositions for treating ischemia or ischemia/reperfusion injury (collectively, IRI) in a subject, comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and wherein the PFKFB3 inhibitor does not inhibit PI3K/AKT/mTOR pathway or HIF1-α.

In one embodiment, the subject is administered an effective amount of the HIF1-α Pathway Inhibitor and the PFKFB3 inhibitor. In one embodiment, the subject is administered an effective amount of the HIF1-α Pathway Inhibitor and the subject has previously been administered the PFKFB3 Inhibitor. In one embodiment, the subject is administered an effective amount of the PFKFB3 Inhibitor and the subject has previously been administered the HIF1-α Pathway Inhibitor.

In some embodiments, a method provided herein (e.g., any of (a)-(c) above), is performed as a prophylactic treatment for IRI. In some embodiments, the subject is at risk of having IRI. In some embodiments, the subject is about to undergo a medical procedure (e.g., surgery, angioplasty, bypass surgery, organ transplantation, or stent surgery).

In some embodiments, the HIF1-α Pathway Inhibitor and the PFKFB3 inhibitor are administered prior to ischemia.

In some embodiments, the HIF1-α Pathway Inhibitor and the PFKFB3 inhibitor are administered during ischemia or prior to reperfusion.

In some embodiments, the HIF1-α Pathway Inhibitor and the PFKFB3 inhibitor are administered during reperfusion.

In some embodiments, the HIF1-α Pathway Inhibitor and the PFKFB3 inhibitor are administered after the ischemia and ischemia/reperfusion.

In some embodiments, the HIF1-α Pathway Inhibitor administered according to a method provided herein is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α Pathway binding polypeptide, or a small molecule HIF1-α Pathway Inhibitor.

In some embodiments, the administered HIF1-α Pathway Inhibitor is silibinin, PX-478 or YC-1, or a salt thereof.

In some embodiments the administered HIF1-α Pathway Inhibitor is ganetespib (ST-9090), phenethyl isothiocyanate, or BAY-87-2243, or a salt thereof.

In some embodiments, the HIF1-α Pathway Inhibitor administered according to a method provided herein is a HIF1-α Inhibitor. In some embodiments, the HIF1-α Inhibitor does not inhibit the PI3K/AKT/mTOR pathway. In some embodiments, the HIF1-α Inhibitor is an antibody or antigen-binding fragment thereof (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

In some embodiments, the administered HIF1-α Inhibitor is antisense oligonucleotide EZN-2968 or nanobody AG-1, AG-2, AG-3, AG-4, AG-5, VHH212, or AHPC.

In some embodiments, the PFKFB3 Inhibitor administered according to a method provided herein is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a PFKFB3 binding polypeptide, or a small molecule PFKFB3 Inhibitor.

In some embodiments, the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

In some embodiments, the administered PFKFB3 Inhibitor is KAN0436151 or KAN0436067, or a salt thereof.

In a particular embodiment, the PFKFB3 inhibitor administered according to the provided methods is AZ67, or a salt thereof.

In some embodiments, the PFKFB3 inhibitor administered according to the provided methods is at least one PFKFB3 inhibitor having the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof. In other embodiments, the PFKFB3 inhibitor administered according to the provided methods is the PFKFB3 inhibitor having the structure of formula AZ44-AZ70 or AZ71, depicted in FIG. 1E, or a salt thereof.

In some embodiments, a method provided herein for treating IRI is performed by co-administering the HIF1-α Pathway Inhibitor and the PFKFB3 inhibitor to the subject.

In some embodiments, the administration of the HIF1-α Pathway Inhibitor and/or the PFKFB3 inhibitor is administered orally. In some embodiments, the administration of the HIF1-α Pathway Inhibitor and/or the PFKFB3 inhibitor is administered, via transmucosal administration, syrup, topical administration, parenteral administration, injection, subdermal administration, rectal administration, buccal administration or transdermal administration.

In some embodiments, the ischemia or ischemia/reperfusion injury is due to a condition selected from: infarction, atherosclerosis, thrombosis, thromboembolism, lipid-embolism, bleeding, stent, surgery, angioplasty, end of bypass during surgery, organ transplantation, or total ischemia.

In some embodiments, the ischemia or ischemia/reperfusion injury is selected from: organ dysfunction, infarct, inflammation, oxidative damage, mitochondrial membrane potential damage, apoptosis, reperfusion-related arrhythmia, cardiac stunning, cardiac lipotoxicity, or ischemia-derived scar formation.

“Organ dysfunction” relates to a condition wherein a particular organ does not perform its expected function. An organ dysfunction develops into organ failure if the normal homeostasis cannot be maintained without external clinical intervention. Methods to determine organ dysfunction are known in the art and include, without limitation, monitorization and scores including sequential organ failure assessment (SOFA) score, multiple organ dysfunction (MOD) score and logistic organ dysfunction (LOD) score.

In some embodiments, the ischemia injury or ischemia/reperfusion injury is due to myocardial infarction.

In some embodiments, the ischemia/reperfusion injury to be prevented and/or treated according to the provided methods occurs in an organ or a tissue of the subject. Organs in which the ischemia/reperfusion injury may occur include, without limitation, brain, heart, kidneys, liver, large intestine, lungs, pancreas, small intestine, stomach, muscles, bladder, spleen, ovaries and testes. In a particular embodiment, the organ is selected from: of heart, liver, kidney, brain, intestine, pancreas, lung, skeletal muscle and combinations thereof. In a more particular embodiment, the organ is heart. Tissues include, without limitation, nerve tissue, muscle tissue, skin tissue and bone tissue.

In some embodiments, treating ischemia or ischemia injury according to a method provided herein comprises reducing one or more symptoms of the ischemia or ischemia injury. In some embodiments, the provided methods result in a reduced apoptosis/destruction (i.e., loss of) of or injury to endothelial cells and/or tissue (e.g., neural tissue); increased survival and/or function of endothelial cells; reduced long-term damage to endothelial cells, and surrounding cells/tissue; decrease of the inflammation in endothelial cells/tissues; reduction in the oxidative stress in endothelial; and increased survival/survival time.

In some embodiments, the provided methods result in a reduced lesion volume, reduced brain inflammatory levels, increased probability of recovery on the mRS score, and/or reduced cytotoxic edema. In some embodiments, the provided methods result in a reduced lesion volume of at least 10%, at least 20%, at least 30%, at least 40%, or at least 50% compared the subject prior to treatment with the HIF1-α Pathway Inhibitor and the PFKFB3 inhibitor. Methods for detecting ischemia and ischemia/reperfusion injury are known in the art and include for example, fluorescein analysis, fluorescent zinc 2,2′-dipicolylamine coordination complex PSVue®794, 99mTc glucarate, and electroretinography.

In some embodiments, 1, 2, 3, 4, 5, or more ischemia reperfusion injury biomarkers such as hyperintense acute reperfusion injury marker (HARM), caspase-3, MMP-2, MMP-9, endothelin-1, leukotrienes B4 and C4; TNFα, IL1, IL6, IL8, PAF ICAM-1, VCAM-1 PECAM-1, and biomarkers of organ/tissue damage) and extent of no reflow phenomenon, of the subject is reduced by at least 10%, at least 20%, at least 30%, at least 40%, or at least 50% compared to the control subject or compared to the subject prior to treatment with the HIF1-α Pathway Inhibitor and the PFKFB3 inhibitor.

In some embodiments, the provide methods reduce ventricular arrythmias in the subject. In some embodiments, the provide methods reduce the extent of no reflow phenomenon in the subject.

In additional embodiments, the provided methods include further administering an additional therapeutic agent to the subject.

The disclosure of each of U.S. Appl. No. 63/189,204, U.S. Appl. No. 63/189,205, U.S. Appl. No. 63/189,206, and U.S. Appl. No. 63/189,207, each filed May 16, 2021, is herein incorporated by reference in its entirety.

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A method of treating a cardiovascular disease in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor;
wherein the PFKFB3 inhibitor does not inhibit PI3K/AKT/mTOR pathway or HIF1-α.

2. The method of claim 1, wherein the subject is administered an effective amount of the HIF1-α Pathway Inhibitor and the PFKFB3 inhibitor.

3. The method of claim 1, wherein the subject is administered an effective amount of the HIF1-α Pathway Inhibitor and wherein the subject has previously been administered the PFKFB3 Inhibitor.

4. The method of claim 1, wherein the subject is administered an effective amount of the PFKFB3 Inhibitor and wherein the subject has previously been administered the HIF1-α Pathway Inhibitor.

5. The method of any one of claims 1-4, wherein the method of any one of claim 1(a)-1(c) is administered as a prophylactic treatment for the cardiovascular disease.

6. The method of any one of claims 1-4, wherein the subject has or is at risk of having the cardiovascular disease.

7. The method of any one of claims 1-4, wherein the subject has or has been diagnosed as having the cardiovascular disease.

8. The method of any one of claims 1-7, wherein the cardiovascular disease is acute coronary syndrome, coronary artery disease, myocardial infarction, coronary heart disease, carditis or cardiomyopathy, an ischemic cardiovascular disease, heart failure, stroke, peripheral vascular disease peripheral arterial disease, and ischemia/reperfusion injury.

9. The method of any one of claims 1-8, wherein the cardiovascular disease is ischemic cardiovascular disease such as myocardial ischemia.

10. The method of any one of claims 1-8, wherein the cardiovascular disease is a peripheral vascular disease or peripheral arterial disease.

11. The method of any one of claims 1-8, wherein the cardiovascular disease is carditis or cardiomyopathy.

12. The method of any one of claims 1-8, wherein the cardiovascular disease is ischemia or ischemia/reperfusion injury.

13. The method of any one of claims 1-12 wherein the administered HIF1-α Pathway Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, MiRNA, dsRNA, ssRNA, and shRNA), a peptibody, a nanobody, a HIF1-α Pathway binding polypeptide, or a small molecule HIF1-α Pathway Inhibitor.

14. The method of any one of claims 1-13 wherein the administered HIF1-α Pathway Inhibitor is silibinin, PX-478 or YC-1, or a salt thereof.

15. The method of any one of claims 1-14 wherein the administered HIF1-α Pathway Inhibitor is ganetespib (ST-9090), phenethyl isothiocyanate, or BAY-87-2243, or a salt thereof.

16. The method of any one of claims 1-15 wherein the administered HIF1-α Pathway Inhibitor is a HIF1-α Inhibitor.

17. The method of claim 16 wherein the HIF1-α Inhibitor is a antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a HIF1-α binding polypeptide, or a small molecule HIF1-α Inhibitor.

18. The method of claim 16 or 17, wherein the administered HIF1-α Inhibitor is antisense oligonucleotide EZN-2968 or nanobody AG-1, AG-2, AG-3, AG-4, AG-5, VHH212, or AHPC.

19. The method of any one of claims 1-18, wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

20. The method of any one of claims 1-19, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

21. The method of any one of claims 1-20, wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

22. The method of any one of claims 1-21, wherein the HIF1-α Pathway Inhibitor and the PFKFB3 inhibitor are co-administered to the subject.

23. The method of any one of claims 1-22, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

24. The method of any one of claims 1-23, wherein the HIF1-α Pathway Inhibitor and the PFKFB3 inhibitor are administered before the onset of one or more symptoms of the cardiovascular disease.

25. The method of any one of claims 1-24, wherein treating the cardiovascular disease comprises delaying the onset of the cardiovascular disease.

26. The method of any one of claims 1-23, wherein the HIF1-α Pathway Inhibitor and the PFKFB3 inhibitor are administered after the onset of one or more symptoms of the cardiovascular disease.

27. The method of claim 26, wherein the method results in one or more symptoms of the cardiovascular disease are reduced in the subject administered the HIF1-α Pathway Inhibitor and the PFKFB3 inhibitor compared to in the subject prior to treatment.

28. The method of claim 27, wherein the one or more reduced symptoms of the cardiovascular disease is indicated by: reduction in apoptosis/destruction (i.e., loss of) of cardiovascular cells and/or tissue (e.g., endothelial cells, cardiomyocytes, and heart); increase in survival and/or function of cardiovascular cells and/or tissue (e.g., endothelial cells, cardiomyocytes, and heart); reduction in long-term damage to cardiovascular cells/tissue and/or to surrounding cells/tissue; decrease of the inflammation in cardiovascular cells/tissues; reduction in the oxidative stress in cardiovascular cells/tissues; or the reduction of levels of serum biomarkers in the subject, such as, TNFα, IL-1β, IL6, MCP1, IL8, PAF, caspase-3, MMP-2, MMP-9, endothelin-1, leukotrienes B4 and C4; ICAM-1, VCAM-1 PECAM-1, creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, transferrin and/or hyperintense acute reperfusion injury marker (HARM).

29. The method of claim 27 or 28, wherein the one or more of the reduction in apoptosis/destruction (i.e., loss of) of cardiovascular cells and/or tissue (e.g., endothelial cells, cardiomyocytes, and heart); increase in survival and/or function of cardiovascular cells and/or tissue (e.g., endothelial cells, cardiomyocytes, and heart); reduction in long-term damage to cardiovascular cells/tissue and/or to surrounding cells/tissue; decrease of the inflammation in cardiovascular cells/tissues; reduction in the oxidative stress in cardiovascular cells/tissues are reduced by at least 10%, at least 20%, at least 30%, at least 40%, or at least 50% compared to in the subject prior to treatment with the HIF1-α Pathway Inhibitor and the PFKFB3 inhibitor.

30. The method according to any one of claims 27-29, wherein the serum levels of at least 1, 2, 3, 4, or 5 of: TNFα, IL-1β, IL6, MCP1, IL8, PAF, caspase-3, MMP-2, MMP-9, endothelin-1, leukotrienes B4 and C4; ICAM-1, VCAM-1 PECAM-1, creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin and/or HARM are reduced by at least 20%, compared to in the subject prior to treatment with the HIF1-α Pathway Inhibitor and the PFKFB3 inhibitor.

31. The method according to any one of claims 27-29, wherein the serum levels of at least 1, 2, 3, 4, or 5 of: TNFα, IL-1β, IL6, MCP1, IL8, PAF, caspase-3, MMP-2, MMP-9, endothelin-1, leukotrienes B4 and C4; ICAM-1, VCAM-1 PECAM-1, and/or HARM are reduced by at least 30%, compared to in the subject prior to treatment with the HIF1-α Pathway Inhibitor and the PFKFB3 inhibitor.

32. The method of any one of claims 1-31, which further comprises administering an additional therapeutic agent to the subject.

33. A method of treating an Acute Coronary Syndrome in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor;
wherein the PFKFB3 inhibitor does not inhibit PI3K/AKT/mTOR pathway or HIF1-α.

34. The method of claim 33, wherein the subject is administered an effective amount of the HIF1-α Pathway Inhibitor and the PFKFB3 inhibitor.

35. The method of claim 33, wherein the subject is administered an effective amount of the HIF1-α Pathway Inhibitor and wherein the subject has previously been administered the PFKFB3 Inhibitor.

36. The method of claim 33, wherein the subject is administered an effective amount of the PFKFB3 Inhibitor and wherein the subject has previously been administered the HIF1-α Pathway Inhibitor.

37. The method of any one of claims 33-36, wherein the method of any one of claim 33(a)-33(c) is administered as a prophylactic treatment for the Acute Coronary Syndrome.

38. The method of any one of claims 33-36, wherein the subject has or is at risk of having the Acute Coronary Syndrome.

39. The method of any one of claims 33-36, wherein the subject has or has been diagnosed as having the Acute Coronary Syndrome.

40. The method of any one of claims 33-39, wherein the Acute Coronary Syndrome is selected from: unstable angina or myocardial infarction (MI) (e.g., Non-ST-segment elevation MI (NSTEMI) and ST-segment elevation MI (STEMI).

41. The method of claim 40, wherein the Acute Coronary Syndrome is unstable angina.

42. The method of claim 40, wherein the Acute Coronary Syndrome is myocardial infarction.

43. The method of claim 40, wherein the Acute Coronary Syndrome is Non-ST-segment elevation MI.

44. The method of claim 40, wherein the Acute Coronary Syndrome is ST-segment elevation MI (STEMI).

45. The method of any one of claims 33-44, wherein the administered HIF1-α Pathway Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor.

46. The method of any one of claims 33-45, wherein the administered HIF1-α Pathway Inhibitor is silibinin, PX-478 or YC-1, or a salt thereof.

47. The method of any one of claims 33-45, wherein the administered HIF1-α Pathway Inhibitor is ganetespib (ST-9090), phenethyl isothiocyanate, or BAY-87-2243, or a salt thereof.

48. The method of any one of claims 33-47, wherein the administered HIF1-α Pathway Inhibitor is a HIF1-α Inhibitor.

49. The method of claim 48, wherein the HIF1-α Inhibitor is a antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

50. The method of claim 48 or 49, wherein the administered HIF1-α Inhibitor is Antisense oligonucleotide EZN-2968 or nanobody AG-1, AG-2, AG-3, AG-4, AG-5, VHH212, or AHPC.

51. The method of any one of claims 33-50, wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

52. The method of any one of claims 33-51, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

53. The method of any one of claims 33-51, wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

54. The method of any one of claims 33-53, wherein the HIF1-α Pathway Inhibitor and the PFKFB3 inhibitor are co-administered to the subject.

55. The method of any one of claims 33-54, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

56. The method of any one of claims 33-55, wherein the HIF1-α Pathway Inhibitor and the PFKFB3 inhibitor are administered before the onset of one or more symptoms of the Acute Coronary Syndrome.

57. The method of any one of claims 33-56, wherein treating the Acute Coronary Syndrome comprises delaying the onset of the Acute Coronary Syndrome.

58. The method of any one of claims 33-57, wherein the HIF1-α Pathway Inhibitor and the PFKFB3 inhibitor are administered after the onset of one or more symptoms of the Acute Coronary Syndrome.

59. The method of any one of claims 33-58, wherein the method results in reduction in one or more symptoms of the Acute Coronary Syndrome the subject administered the HIF1-α Pathway Inhibitor and the PFKFB3 inhibitor compared to in the subject prior to treatment.

60. The method of claim 59, wherein the one or more reduced symptoms of the Acute Coronary Syndrome is indicated by: (A) reduction in angina, chest pain nausea or vomiting, indigestion, dyspnea; sudden, diaphoresis; lightheadedness, dizziness or fainting; unusual fatigue; restlessness or apprehension; (b) a normalized ECG (e.g., reversion of changes in the Q wave and ST segment to normal) or (c) reduced levels of plasma concentrations of cardiac enzymes or other biomarkers (e.g., creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin.

61. The method of claim 59 or 60, wherein the one or more symptoms of the Acute Coronary Syndrome are reduced by at least 10%, at least 20%, at least 30%, at least 40%, or at least 50% compared to in the subject prior to treatment with the HIF1-α Pathway Inhibitor and the PFKFB3 inhibitor.

62. The method according to any one of claims 59-61 wherein at least one of the biomarkers creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin is reduced compared to in the subject prior to treatment with the HIF1-α Pathway Inhibitor and the PFKFB3 inhibitor.

63. The method according to any one of claims 59-62 wherein at least 1, 2, 3, 4, 5 or more of the biomarkers, creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin of the subject are improved by at least 20%, at least 30%, at least 40%, or at least 50% compared to in the subject prior to treatment with the HIF1-α Pathway Inhibitor and the PFKFB3 inhibitor.

64. The method of any one of claims 33-63, which further comprises administering an additional therapeutic agent to the subject.

65. A method of treating myocardial infarction (AD) in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor;
wherein the PFKFB3 inhibitor does not inhibit PI3K/AKT/mTOR pathway or HIF1-α.

66. The method of claim 65, wherein the subject is administered an effective amount of the HIF1-α Pathway Inhibitor and the PFKFB3 inhibitor.

67. The method of claim 65, wherein the subject is administered an effective amount of the HIF1-α Pathway Inhibitor and wherein the subject has previously been administered the PFKFB3 Inhibitor.

68. The method of claim 65, wherein the subject is administered an effective amount of the PFKFB3 Inhibitor and wherein the subject has previously been administered the HIF1-α Pathway Inhibitor.

69. The method of any one of claims 65-68, wherein the method of any one of claim 65(a)-65(c) is administered as a prophylactic treatment for myocardial infarction.

70. The method of any one of claims 65-68, wherein the subject has or is at risk of having myocardial infarction.

71. The method of any one of claims 65-68, wherein the subject has or has been diagnosed as having myocardial infarction (e.g., Non-ST-segment elevation MI (NSTEMI) or ST-segment elevation MI (STEMI)).

72. The method of any one of claims 65-71, wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor.

73. The method of any one of claims 65-72, wherein the administered HIF1-α Pathway Inhibitor is silibinin, PX-478 or YC-1, or a salt thereof.

74. The method of any one of claims 65-72, wherein the administered HIF1-α Pathway Inhibitor is ganetespib (ST-9090), phenethyl isothiocyanate, or BAY-87-2243, or a salt thereof.

75. The method of any one of claims 65-72, wherein the administered HIF1-α Pathway Inhibitor is a HIF1-α Inhibitor.

76. The method of claim 75, wherein the HIF1-α Inhibitor is a antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

77. The method of claim 75 or 76, wherein the administered HIF1-α Inhibitor is Antisense oligonucleotide EZN-2968 or nanobody AG-1, AG-2, AG-3, AG-4, AG-5, VHH212, or AHPC.

78. The method of any one of claims 65-77, wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

79. The method of any one of claims 65-78, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

80. The method of any one of claims 65-78, wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

81. The method of any one of claims 65-80, wherein the HIF1-α Pathway Inhibitor and the PFKFB3 inhibitor are co-administered to the subject.

82. The method of any one of claims 65-81, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

83. The method of any one of claims 65-82, wherein the HIF1-α Pathway Inhibitor and the PFKFB3 inhibitor are administered before the onset of one or more symptoms of myocardial infarction.

84. The method of any one of claims 65-83, wherein treating myocardial infarction comprises delaying the onset of myocardial infarction.

85. The method of any one of claims 65-84, wherein the HIF1-α Pathway Inhibitor and the PFKFB3 inhibitor are administered after the onset of one or more symptoms of myocardial infarction.

86. The method of any one of claims 65-85, wherein the method results in reduction in one or more symptoms of myocardial infarction the subject administered the HIF1-α Pathway Inhibitor and the PFKFB3 inhibitor compared to in the subject prior to treatment.

87. The method of claim 86, wherein the one or more reduced symptoms of myocardial infarction is indicated by:
(a) reduced angina, chest pain; nausea or vomiting, indigestion, dyspnea; diaphoresis; lightheadedness, dizziness or fainting; unusual fatigue; and restlessness;
(b) normalized ECG (e.g., reversion of changes in the Q wave and ST segment to normal);
(c) reduced apoptosis/destruction (i.e., loss of) of or injury to cardiomyocyte cells and/or tissue (e.g., heart); increased survival and/or function of cardiomyocytes and the heart; reduced long-term damage to cardiomyocytes and surrounding cells/tissue; decrease of the inflammation in cardiovascular cells/tissues; reduction in the oxidative stress in cardiovascular cells/tissues; and increased survival/survival time; or
(d) reduced levels reduced levels of plasma MI biomarkers (e.g., creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin.

88. The method of claim 86 or 87, wherein the one or more symptoms of myocardial infarction are reduced by at least 10%, at least 20%, at least 30%, at least 40%, or at least 50% compared to in the subject prior to treatment with the HIF1-α Pathway Inhibitor and the PFKFB3 inhibitor.

89. The method according to any one of claims 86-88, wherein at least one of the plasma MI biomarkers creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin is improved compared to in the subject prior to treatment with the HIF1-α Pathway Inhibitor and the PFKFB3 inhibitor.

90. The method according to any one of claims 86-89, wherein at least 1, 2, 3, 4, or 5 plasma MI biomarkers creatine kinase (CK-MB), troponin, N-terminal pro B-type natriuretic peptide, alpha-1 antitrypsin, C-reactive protein, apolipoprotein A1, apolipoprotein B, creatinine, alkaline phosphatase, and transferrin are reduced by at least 20%, at least 30%, at least 40%, or at least 50% compared to in the subject prior to treatment with the HIF1-α Pathway Inhibitor and the PFKFB3 inhibitor.

91. The method of any one of claims 65-90, which further comprises administering an additional therapeutic agent to the subject.

92. A method of treating heart failure in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and
wherein the PFKFB3 inhibitor does not inhibit PI3K/AKT/mTOR pathway or HIF1-α.

93. The method of claim 92, wherein the subject is administered an effective amount of the HIF1-α Pathway Inhibitor and the PFKFB3 inhibitor.

94. The method of claim 92, wherein the subject is administered an effective amount of the HIF1-α Pathway Inhibitor and wherein the subject has previously been administered the PFKFB3 Inhibitor.

95. The method of claim 92, wherein the subject is administered an effective amount of the PFKFB3 Inhibitor and wherein the subject has previously been administered the HIF1-α Pathway Inhibitor.

96. The method of any one of claims 92-95, wherein the method of any one of claim 92(a)-92(c) is administered as a prophylactic treatment for heart failure.

97. The method of any one of claims 92-95, wherein the subject has or is at risk of having heart failure.

98. The method of any one of claims 92-95, wherein the subject has or has been diagnosed as having heart failure.

99. The method of any one of claims 92-98, wherein the administered HIF1-α Pathway Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor.

100. The method of any one of claims 92-99, wherein the administered HIF1-α Pathway Inhibitor is silibinin, PX-478 or YC-1, or a salt thereof.

101. The method of any one of claims 92-99, wherein the administered HIF1-α Pathway Inhibitor is ganetespib (ST-9090), phenethyl isothiocyanate, or BAY-87-2243, or a salt thereof.

102. The method of any one of claims 92-99, wherein the administered HIF1-α Pathway Inhibitor is a HIF1-α Inhibitor.

103. The method of claim 102, wherein the HIF1-α Inhibitor is a antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

104. The method of claim 102 or 103, wherein the administered HIF1-α Inhibitor is Antisense oligonucleotide EZN-2968 or nanobody AG-1, AG-2, AG-3, AG-4, AG-5, VHH212, or AHPC.

105. The method of any one of claims 92-104, wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

106. The method of any one of claims 92-105, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

107. The method of any one of claims 92-105, wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

108. The method of any one of claims 92-107, wherein the HIF1-α Pathway Inhibitor and the PFKFB3 inhibitor are co-administered to the subject.

109. The method of any one of claims 92-108, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

110. The method of any one of claims 92-109, wherein the HIF1-α Pathway Inhibitor and the PFKFB3 inhibitor are administered before the onset of one or more symptoms of heart failure.

111. The method of any one of claims 92-110, wherein treating heart failure comprises delaying the onset of heart failure.

112. The method of any one of claims 92-111, wherein the HIF1-α Pathway Inhibitor and the PFKFB3 inhibitor are administered after the onset of one or more symptoms of heart failure.

113. The method of any one of claims 92-112, wherein the method results in reduction in one or more symptoms of heart failure the subject administered the HIF1-α Pathway Inhibitor and the PFKFB3 inhibitor compared to in the subject prior to treatment.

114. The method of claim 113, wherein the one or more reduced symptoms of heart failure is indicated by: (a) reduction in shortness of breath, fatigue, weakness, leg swelling, exercise intolerance, elevations in heart and respiratory rates, rales (an indication of fluid in the lungs), edema, jugular venous distension, and an enlarged heart; or (b) reduction of at least one serum biomarker for HF (e.g., plasma hsCRP, IL-1beta and IL-6, and B-type Natriuretic Peptide (BNP)).

115. The method of claim 113 or 114, wherein at least one of the following symptoms is improved in the subject compared to prior to treatment with the HIF1-α Pathway Inhibitor and the PFKFB3 inhibitor: shortness of breath, fatigue, weakness, leg swelling, exercise intolerance, elevations in heart and respiratory rates, rales (an indication of fluid in the lungs), edema, jugular venous distension, and an enlarged heart.

116. The method any one of claims 113-115, wherein at least one of the one of the biomarkers hsCRP, IL-1beta, and IL-6, and B-type Natriuretic Peptide (BNP) is reduced at least 10%, at least 20%, at least 30%, at least 40%, or at least 50% compared to in the subject prior to treatment with the HIF1-α Pathway Inhibitor and the PFKFB3 inhibitor.

117. The method according to any one of claims 113-116 wherein at least 1, 2, or 3 of the biomarkers hsCRP, IL-1beta, IL-6, and B-type Natriuretic Peptide (BNP) is reduced at least 20%, at least 30%, at least 40%, or at least 50% compared to in the subject prior to treatment with the HIF1-α Pathway Inhibitor and the PFKFB3 inhibitor.

118. The method of any one of claims 92-117, which further comprises administering an additional therapeutic agent to the subject.

119. A method of treating stroke in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and
wherein the PFKFB3 inhibitor does not inhibit PI3K/AKT/mTOR pathway or HIF1-α.

120. The method of claim 119, wherein the subject is administered an effective amount of the HIF1-α Pathway Inhibitor and the PFKFB3 inhibitor.

121. The method of claim 119, wherein the subject is administered an effective amount of the HIF1-α Pathway Inhibitor and wherein the subject has previously been administered the PFKFB3 Inhibitor.

122. The method of claim 119, wherein the subject is administered an effective amount of the PFKFB3 Inhibitor and wherein the subject has previously been administered the HIF1-α Pathway Inhibitor.

123. The method of any one of claims 119-122, wherein the method of any one of claim 119(a)-119(c) is administered as a prophylactic treatment for stroke.

124. The method of any one of claims 119-122, wherein the subject has or is at risk of having stroke.

125. The method of any one of claims 119-122, wherein the subject has or has been diagnosed as having stroke.

126. The method of any one of claims 119-125, wherein the administered HIF1-α Pathway Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor.

127. The method of any one of claims 119-126, wherein the administered HIF1-α Pathway Inhibitor is silibinin, PX-478 or YC-1, or a salt thereof.

128. The method of any one of claims 119-126, wherein the administered HIF1-α Pathway Inhibitor is ganetespib (ST-9090), phenethyl isothiocyanate, or BAY-87-2243, or a salt thereof.

129. The method of any one of claims 119-126, wherein the administered HIF1-α Pathway Inhibitor is a HIF1-α Inhibitor.

130. The method of claim 129, wherein the HIF1-α Inhibitor is an antibody or antigen-binding antibody fragment (e.g., a single chain antibody, a single-domain antibody (e.g., a VHH), a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

131. The method of claim 129 or 130, wherein the administered HIF1-α Inhibitor is Antisense oligonucleotide EZN-2968 or nanobody AG-1, AG-2, AG-3, AG-4, AG-5, VHH212, or AHPC.

132. The method of any one of claims 119-131, wherein the administered PFKFB3 Inhibitor is an antibody or antigen-binding antibody fragment (e.g., a single chain antibody, a single-domain antibody, a Fab fragment, F(ab′)2 fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

133. The method of any one of claims 119-132, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

134. The method of any one of claims 119-132, wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

135. The method of any one of claims 119-134, wherein the HIF1-α Pathway Inhibitor and the PFKFB3 inhibitor are co-administered to the subject.

136. The method of any one of claims 119-135, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

137. The method of any one of claims 119-136, wherein the HIF1-α Pathway Inhibitor and the PFKFB3 inhibitor are administered before the onset of one or more symptoms of stroke.

138. The method of any one of claims 119-137, wherein treating stroke comprises delaying the onset of stroke.

139. The method of any one of claims 119-138, wherein the HIF1-α Pathway Inhibitor and the PFKFB3 inhibitor are administered after the onset of one or more symptoms of stroke.

140. The method of any one of claims 119-139, wherein the method results in reduction in one or more symptoms of stroke the subject administered the HIF1-α Pathway Inhibitor and the PFKFB3 inhibitor compared to in the subject prior to treatment.

141. The method of claim 140, wherein the one or more reduced symptoms of stroke is indicated by:
(a) reduced numbness or weakness in the face, arm, or leg, especially on one side of the body; confusion, trouble speaking, or difficulty understanding speech; trouble seeing in one or both eyes; and/or trouble walking, dizziness, loss of balance, or lack of coordination;
(b) reduced lesion volume, reduced brain inflammatory levels, increased probability of recovery on the mRS score, and/or reduced cytotoxic edema;
(c) reduced apoptosis/destruction (i.e., loss of) of or injury to endothelial cells, neural cells, and/or tissue (e.g., neural tissue); increased survival and/or function of vascular endothelial cells and/or neural cells; reduced in long-term damage to vascular endothelial cells, neural cells, and surrounding cells/tissue; decrease of the inflammation in vascular endothelial and/or neural cells/tissues; reduction in the oxidative stress in vascular endothelial and/or neural cells; and increased survival/survival time; or
(D) reduced levels of serum biomarkers for stroke (e.g., E-selectin, ICAM-1, VCAM, and MCP-1) in the subject.

142. The method of claim 140 or 141, wherein the one or more symptoms of stroke are reduced by at least 10%, at least 20%, at least 30%, at least 40%, or at least 50% compared to in the subject prior to treatment with the HIF1-α Pathway Inhibitor and the PFKFB3 inhibitor.

143. The method according to any one of claims 140-142, wherein at least one of the following symptoms are reduced in the subject compared to in the subject prior to treatment with the HIF1-α Pathway Inhibitor and the PFKFB3 inhibitor: numbness or weakness in the face, arm, or leg; confusion, trouble speaking, or difficulty understanding speech; trouble seeing in one or both eyes; and/or trouble walking, dizziness, loss of balance, or lack of coordination.

144. The method according to any one of claims 140-143, wherein at least one serum biomarker for stroke selected from: E-selectin, ICAM-1, VCAM, and MCP-1 is reduced by at least 20%, at least 30%, at least 40%, or at least 50% compared to in the subject prior to treatment with the HIF1-α Pathway Inhibitor and the PFKFB3 inhibitor.

145. The method of any one of claims 119-144, which further comprises administering an additional therapeutic agent to the subject.

146. A method of treating ischemia or ischemia/reperfusion injury in a subject in need thereof comprising:
(a) administering an effective amount of a HIF1-α Pathway Inhibitor and an PFKFB3 inhibitor to the subject;
(b) administering an effective amount of a HIF1-α Pathway Inhibitor to the subject, wherein the subject has previously been administered a PFKFB3 Inhibitor; or
(c) administering an effective amount of a PFKFB3 Inhibitor to the subject, wherein the subject has previously been administered a HIF1-α Pathway Inhibitor; and
wherein the PFKFB3 inhibitor does not inhibit PI3K/AKT/mTOR pathway or HIF1-α.

147. The method of claim 146, wherein the subject is administered an effective amount of the HIF1-α Pathway Inhibitor and the PFKFB3 inhibitor.

148. The method of claim 146, wherein the subject is administered an effective amount of the HIF1-α Pathway Inhibitor and wherein the subject has previously been administered the PFKFB3 Inhibitor.

149. The method of claim 146, wherein the subject is administered an effective amount of the PFKFB3 Inhibitor and wherein the subject has previously been administered the HIF1-α Pathway Inhibitor.

150. The method of any one of claims 146-149, wherein the method of any one of claim 146(a)-146(c) is administered as a prophylactic treatment for ischemia or ischemia/reperfusion injury.

151. The method of any one of claims 146-149, wherein the subject has or is at risk of having ischemia or ischemia/reperfusion injury.

152. The method of any one of claims 146-149, wherein the subject has or has been diagnosed as having ischemia or ischemia/reperfusion injury.

153. The method of any one of claims 146-153, wherein the administered HIF1-α Pathway Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, a Dicer substrate, dsRNA, ssRNA, and shRNA), a peptibody, a nanobody, a HIF1-α Pathway binding polypeptide, or a small molecule HIF1-α Pathway Inhibitor.

154. The method of any one of claims 146-153, wherein the administered HIF1-α Pathway Inhibitor is silibinin, PX-478 or YC-1, or a salt thereof.

155. The method of any one of claims 146-153, wherein the administered HIF1-α Pathway Inhibitor is ganetespib (ST-9090), phenethyl isothiocyanate, or BAY-87-2243, or a salt thereof.

156. The method of any one of claims 146-153, wherein the administered HIF1-α Pathway Inhibitor is a HIF1-α Inhibitor.

157. The method of claim 156, wherein the HIF1-α Inhibitor is an antibody or antigen-binding antibody fragment (e.g., a single chain antibody, a single-domain antibody (e.g., a VHH), a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, miRNA, dsRNA, ssRNA, and shRNA), a peptibody, a nanobody, a HIF1-α binding polypeptide, or a small molecule HIF1-α Inhibitor.

158. The method of claim 156 or 157, wherein the administered HIF1-α Inhibitor is Antisense oligonucleotide EZN-2968 or nanobody AG-1, AG-2, AG-3, AG-4, AG-5, VHH212, or AHPC.

159. The method of any one of claims 146-158, wherein the administered PFKFB3 Inhibitor is an antibody or antigen-binding antibody fragment (e.g., a single chain antibody, a single-domain antibody, a Fab fragment, F(ab′)₂ fragment, Fd fragment; Fv fragment, scFv, dAb fragment, or another engineered molecule, such as a diabody, triabody, tetrabody, minibody, and a minimal recognition unit), a nucleic acid molecule (e.g., an aptamer, antisense molecule, ribozyme, MiRNA, dsRNA, ssRNA, and shRNA), a peptibody, a nanobody, a PFKFB3 binding polypeptide, or a small molecule PFKFB3 Inhibitor.

160. The method of any one of claims 146-159, wherein the administered PFKFB3 Inhibitor is BrAcNHEtOP (N-bromoacetylethanolamine phosphate), PFK15 (1-(4-pyridinyl)-3-(2-quinolinyl)-2-propen-1-one), or PFK-158 ((E)-1-(4-Pyridinyl)-3-[7-(trifluoromethyl)-2-quinolinyl]-2-propen-1-one), or a salt thereof.

161. The method of any one of claims 146-159, wherein the administered PFKFB3 Inhibitor: (a) is KAN0436151 or KAN0436067, or a salt thereof; (b) has the structure of formula 1-53 or 54, PQP, N4A, YN1, PK15, PFK-158, YZ29, Compound 26, KAN0436151, KAN0436067, or BrAcNHErOP, depicted in FIG. 1A-1C or 1D, or a salt thereof; (c) has the structure of formula AZ44-AZ70 or AZ71, depicted in FIG. 1E, or a salt thereof; or (d) is AZ67, or a salt thereof.

162. The method of any one of claims 146-161, wherein the HIF1-α Pathway Inhibitor and the PFKFB3 inhibitor are co-administered to the subject.

163. The method of any one of claims 146-162, wherein the administration of the HIF1-α Pathway Inhibitor and/or the PFKFB3 inhibitor administration is oral, parenteral, orthotopic, intradermal, subcutaneous, intramuscular, intraperitoneal, intranasal, intratumoral, or intravenous.

164. The method of any one of claims 146-163, wherein the ischemia or ischemia/reperfusion injury is due to a condition selected from: infarction, atherosclerosis, thrombosis, thromboembolism, lipid-embolism, bleeding, stent, surgery, angioplasty, end of bypass during surgery, organ transplantation, or total ischemia.

165. The method of any one of claims 146-163, wherein the ischemia or ischemia/reperfusion injury is ischemia/reperfusion injury is selected from: organ dysfunction, infarct, inflammation, oxidative damage, mitochondrial membrane potential damage, apoptosis, reperfusion-related arrhythmia, cardiac stunning, cardiac lipotoxicity, or ischemia-derived scar formation.

166. The method of any one of claims 146-165, wherein ischemia/reperfusion injury is due to myocardial infarction.

167. The method of any one of claims 146-166, wherein the HIF1-α Pathway Inhibitor and the PFKFB3 inhibitor are administered:
(a) during ischemia or prior to reperfusion;
(b) during reperfusion; or
(c) after the ischemia and ischemia/reperfusion.

168. The method of any one of claims 146-167, wherein treating ischemia or ischemia/reperfusion injury comprises delaying the onset of ischemia or ischemia/reperfusion injury.

169. The method of any one of claims 146-168, wherein the HIF1-α Pathway Inhibitor and the PFKFB3 inhibitor are administered after the onset of one or more symptoms of ischemia or ischemia/reperfusion injury.

170. The method of any one of claims 146-169, wherein the method results in reduction in one or more symptoms of ischemia or ischemia/reperfusion injury the subject administered the HIF1-α Pathway Inhibitor and the PFKFB3 inhibitor compared to in the subject prior to treatment.

171. The method of claim 170, wherein the one or more reduced symptoms of ischemia or ischemia/reperfusion injury is indicated by: (a) reduced apoptosis/destruction (i.e., loss of) of or injury to endothelial cells and/or tissue (e.g., neural tissue); increased survival and/or function of endothelial cells; reduced long-term damage to endothelial cells, and surrounding cells/tissue; decrease of the inflammation in endothelial cells/tissues; reduction in the oxidative stress in endothelial; and increased survival/survival time; (b) reduced levels of at least one ischemia/reperfusion biomarker (e.g., caspase-3, MMP-2, MMP-9, endothelin-1, leukotrienes B4 and C4; TNFα, IL1, IL6, IL8, PAF, ICAM-1, VCAM-1 PECAM-1, and HARM; or (c) reduced extent of no reflow phenomenon in the subject.].

172. The method of claim 170 or 171, wherein the one or more symptoms of ischemia or ischemia/reperfusion injury are reduced by at least 10%, at least 20%, at least 30%, at least 40%, or at least 50% compared to in the subject prior to treatment with the HIF1-α Pathway Inhibitor and the PFKFB3 inhibitor.

173. The method according to any one of claims 170-172, wherein the extent of no reflow phenomenon is reduced in the subject.

174. The method according to any one of claims 170-173 wherein the level of at least 1, 2, 3, 4, or 5, ischemia/reperfusion biomarker selected from caspase-3, MMP-2, MMP-9, endothelin-1, leukotrienes B4 and C4; TNFα, IL1, IL6, IL8, PAF, ICAM-1, VCAM-1 PECAM-1, and HARM is reduced in the subject by at least 20%, at least 30%, at least 40%, or at least 50% compared to in the subject prior to treatment with the HIF1-α Pathway Inhibitor and the PFKFB3 inhibitor.

175. The method of any one of claims 146-174, which further comprises administering an additional therapeutic agent to the subject.
